(19) United States
(12) Patent Application Publication
(10) Pub. No.: US 2009/0253777 A1
(43) Pub. Date: Oct. 8, 2009

(54) Methods of diagnosing and treating hyperproliferative disorders

(76) Inventors: Hartmut M. Hanauske-Abel, Edgewater, NJ (US); Myung-Hee Park, Potomac, MD (US); Edith C. Wolff, Bethesda, MD (US); Debra Sheldon-Heller, Closter, NJ (US); Bernadette M. Cracciolo, Edgewater, NJ (US); Anthony Popowicz, Farmingville, NY (US); Paul M.J. Clement, Heverlee (BE)

Correspondence Address: KLAUBER & JACKSON, 411 HACKENSACK AVENUE, HACKENSACK, NJ, 07601, US

(21) Appl. No.: 10/527,453

(22) PCT Filed: Sep. 13, 2003
(86) PCT No.: PCT/US03/28742

§ 371 Date: Mar. 15, 2006

Related U.S. Application Data
(60) Provisional application No. 60/410,778, filed on Sep. 13, 2002.
(60) Provisional application No. 60/417,738, filed on Oct. 10, 2002.
(60) Provisional application No. 60/424,978, filed on Nov. 8, 2002.

(51) Int. Cl. A61K 31/7088; C07K 16/18; G01N 33/574; G01N 33/566; C12N 5/06

(52) U.S. Cl. 514/44 R; 530/387.9; 435/7.23; 436/501; 435/375

(57) ABSTRACT

The invention relates to compositions and methods for diagnosing and treating hyperproliferative disorders using ligands which specifically recognize the hypusine and/or folate binding region of mature eukaryotic translation initiation factor 5A (hypusine-containing eIF-5A). The invention further relates to methods of identifying molecules which displace immunoreagents binding to mature eIF-5A. Such agents are useful for treating hyperproliferative disorders.

### GOVERNMENT RIGHTS CLAUSE

This invention was made in part in the course of research performed at the National Institutes of Health. The U.S. government may have certain rights in this invention.

### FIELD OF THE INVENTION

The invention relates generally to the field of cellular proliferation, and more particularly, to the detection, measurement and control of aberrant cellular proliferation, as exemplified by cancer and other related disorders.

### BACKGROUND OF THE INVENTION

Normal tissue homeostasis is achieved by an intricate balance between the rate of cell proliferation and cell death. Disruption of this balance either by increasing the rate of cell proliferation or decreasing the rate of cell death can result in the abnormal growth of cells and is a major event in the development of cancer.

Cell proliferation involves many cellular processes including transcription and translation of proteins. Steady-state mRNA levels are maintained by a balance between recruitment to ribosomes for translation and degradation by nucleolytic enzymes. Eukaryotic initiation factor 5A (hypusine containing eIF-5A) has been implicated in several steps of RNA metabolism including both translation and mRNA degradation.

Hypusine-containing eIF-5A is a highly conserved protein encoded in the genomes of eukaryotes and archaebacteria [Park, M. H., Wolff; E. C., Folk, J. E. Biofactors (1993), 4:95-104; Park, M. H., Lee, Y. B., Joe, Y. A.; (1997), Biol Signals, (1997), 6: 115-123; Park, M. H., Wolff, E. C., and Folk, J. E., (1993), Trends Biochem SCI 18:475-479; Chen and Liu (1997) Biol. Signals 6:105-109]. Yeast and mammalian eIF5A proteins are 63% identical, indicating the importance of this protein in basic cellular processes [Schnier, et al. (1991) Mol. Cell. Biol. 11:3105-3114]. Originally purified from ribosomes of rabbit reticulocyte lysates [Kemper, et al. (1976) J. Biol. Chem. 251:5551-5557], hypusine-containing eIF-5A was described as a translation initiation factor due to its ability to stimulate the synthesis of methionyl-puromycin in vitro [Benne and Hershey (1978) J. Biol. Chem. 253:3078-3087; Park, et al. (1993) Biofactors 4:95-104). However, depletion of this factor in yeast caused only a small (30%) reduction in the protein synthesis rate [Kang and Hershey (1994) J. Biol. Chem. 269:3934-3940].

Alternatively, hypusine-containing eIF-5A may be involved in the translation of a specific subset of mRNAs, for example, those involved in the cell cycle progression [G1/S transition; Park, et al. (1993) Biofactors 4:95-104; Park, et al. (1997) Biol. Signals 6:115-123]. Expression of hypusine-containing eIF-5A has also been correlated with cell proliferation: an increase in G1-arrested cells is observed after depletion of this factor in yeast [Kang and Hershey (1994) J. Biol. Chem. 269:3934-3940). Hypusine-containing eIF-5A expression is induced in activated human T lymphocytes [Cooper, H. L., Proc. Natl. Acad. Sci. USA (1993), 80: 1854-1857; Bevec, et al. (1994) Proc. Natl. Acad. Sci. USA 91:10829-10833].

A critical modification to eIF-5A is the formation of a single hypusine residue from a single, specific lysine. [Park, M. H., Wolff, E. C., Folk, J. E. Biofactors (1993), 4:95-104; Park, M. H., Lee, Y. B., Joe, Y. A.; (1997); Park, M. H., Wolff, E. C., and Folk, J. E., (1993), Trends Biochem SCI 18:475-479; Chen and Liu (1997) Biol. Signals 6:105-109; Park, et al. (1997) Biol. Signals 6:115-123]. Hypusine is formed via two consecutive posttranslational modifications of said lysine, catalyzed by deoxyhypusine synthase and by deoxyhypusine hydroxylase. To date, mature eIF-5A is the only protein in nature known to contain hypusine. Hypusination causes distinct measureable changes within the conformation of eIF-5A, possibly associated with the emergence of novel antigenic epitopes in addition to the hypusine residue itself (Joao, H. C. et al. (1995), Biochemistry 34: 14703-14711). These changes, and especially the de novo formation of the hypusine side chain make eIF-5A a molecule with unique immunogenic characteristics. The current application rests on this particular fact. Hypusination of eIF-5A is essential for proliferation of eukaryotic cells, as stringently shown by targeted mutations in yeast. Strains in which hypusination of eIF-5A is blocked, by mutation of the target lysine (K51R) or deletion of the deoxyhypusine synthase gene, do not prolilferate and are not viable [Schnier, et al. (1991) Mol. Cell. Biol. 11:3105-3114; Sasaki, et al. (1996) FEBS Lett. 384:151-154; Park, et al. (1998) J. Biol. Chem. 273:1677-1683]. Similarly, pharmacologic inhibitors of hypusination block proliferation in mammalian cell lines [Tschank, G. et al. (1987), Eur. J. Cell Biol. 43: Supplement 17: 60; Jahner et al., (1990), Proc. Amer. Assoc. Cancer Res. 31: 417; Hanauske-Abel, et al. (1994) Biochim. Biophys. Acta 1221:115-124; McCaffrey T. a. et al. (1995), J. Clin. Invest 95: 446-455; Clement, et al. (2002) Int. J. Cancer 100:491-498; Nishimura, et al. (2002) Biochem. J. 363:761-768; Park, et al. (1994) J. Biol. Chem. 269:27827-27832; Chen, et al. (1996) Cancer Lett. 105:233-239; Shi, et al. (1996) Biochim. Biophys. Acta 1310:119-126]. Moreover, mRNAs encoding enzymes critical for proliferation, disappear from, and reappear at, polysomes in concert with inhibition and disinhibition of the hypusine-forming deoxyhypusyl hydroxylase [Hanauske-Abel, et al. (1995) FEBS Lett. 366:92-98]. The N-terminal acetylated serine residue of eIF-5A is phosphorylated [Kang, et al. (1993) J. Biol. Chem. 268:14750-14756; Klier, et al. (1993) FEBS Lett. 334:360-364]; however, phosphorylation is not essential for hypusine-containing eIF-5A function in vivo.

Hypusine containing eIF-5A may participate in the nucleocytoplasmic trafficking of the HIV-1 Rev protein/RRE complex [Ruhl, et al. (1993) J. Cell Biol. 123:1309-1320; Bevec, et al. (1996) Science 271:1858-1860; Bevec and Hauber (1997) Biol. Signals 6:124-133; Liu, et al. (1997) Biol. Signals 6:166-174; Hofmann, et al. (2001) J. Cell Biol. 152:895-910]; however, hypusine-containing eIF-5A does not directly interact with REV in a nuclear export signal-dependent manner [Henderson and Percipalle (1997) J. Mol. Biol. 274:693-707] Moreover, the cellular localization of hypusine-containing eIF-5A is not consistent with a Rev eIF-5A interaction [Shi, et al. (1997) Biol. Signals 6:143-149]. In mammalian cells, hypusine-containing eIF-5A is mainly cytoplasmic with a fraction associated with the endoplasmic reticulum (ER) membrane through ribosomes [Shi, et al. (1996) Exp. Cell Res. 225:348-356]. Its cytoplasmic localization, combined with its interaction with the ribosomal protein L5 [Schatz, et al. (1998) Proc. Natl. Acad. Sci. USA 95:1607-1612], further indicates a role for hypusine-containing eIF-5A in translation.

A yeast mutant harboring a temperature-sensitive allele of eIF-5A, tif51A, exhibits a defect in mRNA decay, accumulating uncapped mRNAs at the restrictive temperature. In addition, this strain shows an 30% decrease in protein synthesis at high temperature [Zuk and Jacobson (1998) EMBO J. 17:2914-2925]. Furthermore, poly(A)-binding protein and protein kinase C are multicopy suppressors of tif51A-1, indicating a role for eIF-5A in RNA metabolism, including translation, mRNA decay, and ribosome biogenesis [Valentini, et al. (2002) Genetics 160:393-405].

The effects of hyperproliferative disorders such as cancer are catastrophic. Cancer causes over half a million deaths per year in the United States alone. Conventional strategies for the treatment of cancer include chemotherapy, radiotherapy, surgery or combinations thereof, however further advances in these strategies are limited by lack of specificity and excessive toxicity to normal tissues. Generally, both standard chemotherapy and radiotherapy, as well as transfer of genetic material into cells, have limitations; there clearly remains a need for improved strategies of anti-cancer and anti-proliferative cell therapy.

In particular, there is a need to decrease the level of undesirable cellular proliferation beyond that provided by traditional therapies.

### SUMMARY OF THE INVENTION

In its broadest aspect, the present invention relates to eukaryotic translation initiation factor 5A (hypusine-containing eIF-5A) and to ligands that recognize the hypusine containing eIF-5A and/or the folate-binding region of eIF-5A. These ligands may be used for diagnostic and/or therapeutic purposes for identification and/or treatment of conditions wherein unwanted cellular proliferation is prevalent and wherein it is desirous to control such unwanted cellular proliferation. The invention also relates to methods of screening for agents that are inhibitors of cellular proliferation or for agents that inhibit the multiplication of retroviruses that rely on host hypusine-containing eIF-5A for replication. More particularly, the invention relates to the use of these agents to inhibit the biological activity of hypusine-containing eIF-5A required for cellular proliferation. The ligands of the invention or the novel agents identified by the methods described herein may be used alone in the treatment of conditions wherein cellular proliferation is undesirable, or they may be used as adjunct therapy with other agents to treat cancers, or retroviral infections, or other hyperproliferative conditions wherein inhibition of cellular proliferation is desired. The instant invention also provides for pharmaceutical compositions comprising, and methods of using the agents of the present invention for treatment of cancer or hyperproliferative disorders.

Eukaryotic translation initiation factor 5A (hypusine-containing eIF-5A) is involved in the cellular machinery that controls the onset of DNA replication and initiates proliferation of cells. However, the impact of ligands to the hypusine-binding region of eIF-5A for use in the diagnosis and/or treatment of hyperproliferative conditions and the potential use of ligands of the folate-binding region of eIF-5A for therapeutic uses for inhibition of unwanted cellular proliferation was not realized until the time of the present invention.

It is an object of the present invention to provide ligands specific for the hypusine and/or folate-binding region of eIF-5A and to utilize these ligands for diagnostic and/or therapeutic purposes, and to screen for novel agents for treatment of cancer, retroviral infections and other hyperproliferative disorders.

Accordingly, a first aspect of the present invention provides for a ligand recognizing and/or binding to the hypusine region of mature eukaryotic initiation factor 5A. Said hypusine region is defined as extending 15 residues outward from the position of the lysine that gives rise to hypusine, ie. residue 50 in human eIF-5A, to both the carboxy and amino terminals, ie. residues 35 to 65 of human eIF-5A, as set forth in amino acid sequence as in SEQ ID NO: 1 (NP_{—}001961; eiF-5A-1) and SEQ ID NO: 2 (NP_{—}065123; eIF-5A-1). Said ligand binding to the hypusine region of mature eIF-5A in biological samples results in a detectable signal for identification of hypusine-containing eIF-5A, and its hypusine-containing fragments.

In a preferred embodiment, the present invention provides for antibodies, or derivatives or fragments thereof, as ligands which specifically bind to a hypusine containing eIF-5A molecule, or to a non-hypusine containing eIF-5A molecule in an amount not greater than 5% of the extent binding to the hypusine containing eIF-5A molecule. In a further preferred embodiment, the antibodies, or derivatives or fragments thereof, specifically bind to a human hypusine-containing eIF-5A molecule, if said eIF-5A molecule contains hypusine. The antibodies may be polyclonal or monoclonal. They may be single chain antibodies. They may be chimeric antibodies. They may be Fab fragments or soluble components thereof. They may be human or humanized. They may be produced in other animals, including but not limited to horses, goats, sheep, mice, rats, rabbits and guinea pigs.

A second aspect of the invention provides for use of the ligands, eg. the antibodies described herein, to diagnose and/or treat conditions wherein unwanted cellular proliferation is prevalent, for example, in many cancers or other hyperproliferative disorders, such as psoriasis and restenosis. In one embodiment, the ligands of the present invention may be used to treat diseases or disorders whereby there is an increase in proliferation of unwanted immune cells, such as lymphocytes or macrophages. In particular, the ligands may be utilized as immunosuppressants for treatment of conditions such as autoimmune disorders, for treatment of transplant patients to prevent tissue or organ rejection, or for treatment of conditions such as rheumatoid arthritis or multiple sclerosis.

In a preferred embodiment, the invention provides for a method for distinguishing proliferating cells from non-proliferating cells in a specimen of biological fluid or tissue, said method comprising:
a) Processing a specimen of biological fluid or tissue to yield a mixture of cells, said mixture consisting of proliferating and non-proliferating cells present in the biological fluid or tissue; and
b) Treating said mixture of cells with a fixing agent to permeabilize and fix the cells; and
c) Reacting said cells with a ligand, wherein said ligand recognizes the hypusine-containing region of eIF-5A; and
d) Separating said fixed cells from unreacted ligand from Step c; and
e) Detecting said ligand remaining within the fixed cells, whereby detection of said ligand is accomplished by use of a reagent selected from the group consisting of a radiolabel, an enzyme, a chromophore and a fluorescer,
wherein the detecting of said ligand indicates the presence of proliferating cells.

It should be obvious to one skilled in the art that steps a) through e) for distinguishing proliferating from non-proliferating cells should not be limited to particulars or the order in which they appear, that is, steps a) through e) may be modified in execution to optimize the conditions for particular measurements of cellular proliferation.

In a further preferred embodiment, the specimen may be deposited on a solid support and the ligand within the cells of the specimen may be detected using a microscope. In yet another preferred embodiment, the specimen may be maintained in suspension and the ligand within the cells of the speciman may be detected using a flow cytometer. In a further preferred embodiment, the ligand is an antibody specific for hypusine-containing eIF-5A.

In another preferred embodiment, the method of detection of the ligand may be accomplished through use of various detection methods, including, but not limited to use of radiolabels, enzymes, and other chromophores or fluorescent reagents that allow for detection using microscopic techniques or through use of flow cytometric techniques known to those skilled in the art.

A third aspect of the invention provides for a method of diagnosing a hyperproliferative disorder, or a disorder in which determination of proliferating cells is highly desirable for diagnostic and/or therapeutic purposes, comprising contacting a biological sample with a ligand and detecting said ligand bound to hypusine-containing eIF-5A in the sample, wherein the detection of ligand bound to hypusine-containing eIF-5A is indicative of a hyperproliferative disorder.

In a preferred embodiment, the ligand is an antibody, or a derivative or fragment thereof, which specifically binds to a hypusine-containing eIF-5A molecule, or to a hypusine-deficient eIF-5A molecule in an amount not greater than 5% of the extent binding to the hypusine containing eIF-5A molecule. In another preferred embodiment, the antibody or a derivative or fragment thereof specifically binds to a human hypusine-containing eIF-5A molecule, and the binding occurs only if said eIF-5A contains hypusine.

A fourth aspect of the invention provides for a method of diagnosing intraepithelial neoplasia comprising contacting a biological sample with a ligand and detecting said ligand bound to hypusine-containing eIF-5A in the sample, wherein the detection of ligand bound to hypusine-containing eIF-5A is indicative of local neoplasia.

In a preferred embodiment, the biological sample is a biopsy containing epithelium. In another preferred embodiment, the ligand is an antibody, or a derivative or fragment thereof, which specifically binds to a hypusine-containing eIF-5A molecule, and to a hypusine-deficient eIF-5A molecule in an amount of up to about 5% of the extent binding to the hypusine-containing eIF-5A molecule. In another preferred embodiment, the antibody or a derivative or fragment thereof specifically binds to a human hypusine-containing eIF-5A molecule, and the binding occurs only if said eIF-5A contains hypusine.

A fifth aspect of the invention provides for a method for determining in a biological sample the concentration of hypusine-containing eIF-5A and/or of hypusine, either as a free amino acid or bound within the hypusine region of eIF-5A, wherein said hypusine region is located between amino acid residues 35 to 65 of the human eIF-5As as set forth in SEQ ID NOs: 1 and 2, comprising:
a. contacting said sample with a ligand under conditions wherein said ligand can form a complex with hypusine contained in the sample either as a free amino acid or bound within the hypusine region of eIF-5A; and
b. determining the amount of hypusine-containing eIF-5A and of hypusine bound by said ligand by detecting the amount of complex formed, wherein said detecting is accomplished by use of a radiolabel, an enzyme, a chromophore or a flourescer.

In a preferred embodiment, the ligand is an antibody, or a derivative or fragment thereof, which specifically binds to a hypusine-containing eIF-5A molecule, and to a hypusine-deficient eIF-5A molecule in an amount of up to about 5% of the extent binding to the hypusine-containing eIF-5A molecule. In another preferred embodiment, the antibody or a derivative or fragment thereof specifically binds to a human hypusine-containing eIF-5A molecule, and the binding occurs only if said eIF-5A contains hypusine.

A sixth aspect of the invention provides for a method for inhibiting in a cell the biological activity of the hypusine region of eIF-5A that corresponds to amino acid residues 35 to 65 of human eIF-5A as set forth in SEQ D NOs: 1 and 2, comprising:
a. introducing into said cell of a patient in need of such treatment a nucleic acid molecule encoding an antibody homologue, or a derivative or fragment thereof; wherein said antibody homologue, derivative or fragment thereof is specifically reactive to the hypusine region of hypusine-containing eIF-5A; and
b. wherein said antibody homologue is expressed intracellularly and binds to said hypusine region intracellularly thereby inhibiting the biological activity of the hypusine region of hypusine-containing eIF-5A.

In a preferred embodiment, the antibody homologue is a single chain Fv fragment (scFv). In another preferred embodiment, the nucleic acid molecule is a recombinant expression vector selected from the group consisting of, but not limited to, viral vectors and plasmid vectors. In a yet further preferred embodiment, an antibody homologue, such as a single chain Fv fragment, is expressed within an intracellular compartment of a cell, to inhibit expression of a hypusine-containing eIF-5A protein. Preferably, the cell is a cancerous mammalian cell and the protein is hypusine-containing eIF-5A. Intracellular binding of the antibody homologue to the hypusine-containing eIF-5A protein inhibits cell proliferation and cell survival.

A seventh aspect of the invention provides for a method of identifying by high throughput screening a therapeutic agent that decreases the biological activity of the hypusine region of eIF-5A, comprising contacting hypusine-containing eIF-5A with an agent and detecting the binding of an antibody as described herein, or a derivative of fragment thereof, to hypusine-containing eIF-5A. In a preferred embodiment, the antibody specifically binds to a human hypusine containing eIF-5A molecule, and the binding occurs only if said eIF-5A contains hypusine. The method comprises contacting hypusine-containing eIF-5A with an agent and determining whether said agent displaces the binding of a ligand to the eIF-5A region containing hypusine, said ligand being represented by a hypusine region specific anti-eIF-5A antibody. In one embodiment, the method comprises the steps of:
a) Preparing a purified preparation of hypusine-containing eIF-5A;
b) Attaching the purified hypusine-containing eIF-5A to a solid support;
c) Contacting the hypusine-containing eIF-5A on the solid support in the presence of a test compound under conditions which allow binding of the test compound;
d) Washing to remove non-bound test compound;
e) Detecting the amount of hypusine-containing eIF-5A with an antibody or fragment thereof, wherein said detecting may be accomplished using a second antibody which is labeled with a radioactive isotope or an enzyme or chromophore; and
f) Comparing the amount of labeled second antibody bound to a sample without test compound; wherein the amount of labeled antibody bound correlates inversely with the potential of the test compound for decreasing the biological activity of the hypusine region of hypusine-containing eIF-5A.

It should be obvious to one skilled in the art that steps a) through e) for distinguishing proliferating from non-proliferating cells should not be limited to particulars or the order in which they appear, that is, steps a) through e) may be modified in execution to optimize the conditions for particular measurements of cellular proliferation.

In another preferred embodiment, the high throughput screening of the biological activity of the hypusine region of hypusine-containing eIF-5A is directed at cell proliferation. In yet another preferred embodiment, the high throughput screening of the biological activity of the hypusine region of hypusine-containing eIF-5A is directed at retroviral multiplication.

An eighth aspect of the invention provides for methods of using such agents to treat hyperproliferative disorders or to suppress the immune response in conditions where unwanted proliferation of immune cells is prevalent. Examples of such conditions include, but are not limited to, autoimmune diseases, exemplified by rheumatoid arthritis, multiple sclerosis and type I diabetes, or treatment of transplant patients to prevent rejection of transplanted tissues or organs. In a preferred embodiment, such agents are provided in the form of a pharmaceutical composition with a pharmaceutically acceptable carrier for treatment of subjects in need of such therapy. In another preferred embodiment the subject to be treated is a mammal, preferably a human, although use of the agents for treatment of such conditions in other mammals is also conceived.

A ninth aspect of the invention provides for a method of assessing the outcome of anti-cancer therapy, said method comprising:
a) obtaining a sample or tissue biopsy from a subject diagnosed with a cancer or a hyperproliferative disorder prior to the start of therapy (pre-therapy);
b) obtaining a sample or tissue biopsy after cessation of therapy (post therapy);
c) incubating the pre and post therapy samples with an antibody specific for hypusine-containing eIF-5A;
d) washing to remove unbound antibody;
e) incubating with a second antibody which is radiolabeled or labeled with an enzyme;
f) washing to remove unbound second antibody;
g) measuring the amount of second antibody bound by monitoring the amount of radiolabel present or enzyme present; wherein the amount of the second antibody bound inversely correlates with the effectiveness of the therapy.

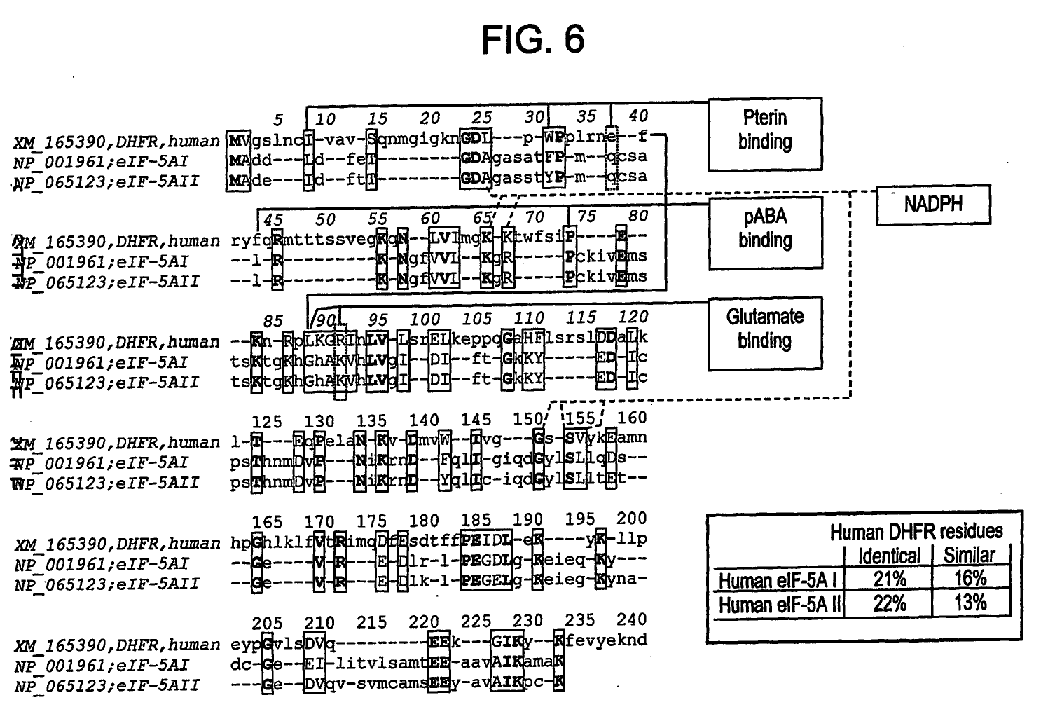

A tenth aspect of the invention provides for a ligand specific for the folate-binding region of eukaryotic translation initiation factor 5A, wherein said folate-binding region comprises at least one residue motif common to eIF-5A and to the bacterial and human dihydrofolate reductases as shown in FIG. 6. In a preferred embodiment, the ligand is selected from the group consisting of an analog of folate, derivatives thereof and fragments thereof, which specifically bind to an eIF-5A molecule only if said eIF-5A contains a folate-binding region.

An eleventh aspect of the invention provides for a method for identifying folate derivatives that are inhibitors of proliferation yet do not inhibit folate-dependent enzymes, comprising placing the folate derivatives under investigation in contact with an eIF-5A molecule containing a folate-binding region, and measuring the extent, if any, to which said folate derivatives specifically bind said eIF-5A molecule. In a preferred embodiment, the folate derivatives under investigation are placed in contact with said eIF-5A molecule containing a folate-binding region, and measuring the extent to which said folate derivatives successfully bind with said eIF-5A molecule. The method for identifying such folate derivatives may be done in a competitive or non-competitive assay format, as known to those skilled in the art.

A twelfth aspect of the invention provides for a method for inhibiting in a cell the biological activity of the folate-binding region of eIF-5A, said folate binding region comprising residue motifs as set forth in FIG. 6, and further comprising introducing into said cell a low-molecular weight molecule that attaches to the folate-binding region of eIF-5A to cause the binding of said low-molecular weight molecule with said eIF-5A, and to thereby inhibit the biological activity of eIF-5A in the translational control of gene expression required for cell proliferation.

Other objects and advantages will become apparent from a review of the ensuing detailed description and attendant claims taken in conjunction with the following illustrative drawings. All references cited in the present application are incorporated herein in their entirety.

### BRIEF DESCRIPTION OF THE VISUAL MATERIALS

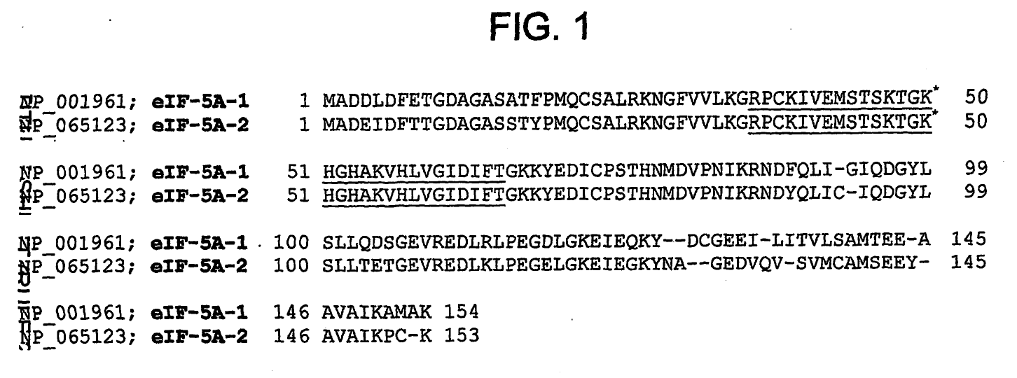

FIG. 1: Receptor Site Sequence for Ligands of Eukaryotic Translation Initiation Factor 5A Containing Hypusine

Deduced amino acid sequences for the two presently known molecular variations (eIF-5A-1: SEQ ID NO: 1 and eIF-5A-2: SEQ ID NO: 2) of eIF-5A in humans, each one encoded by a distinct gene. The hypusine residue is formed from, and occurs in the position of, the labeled lysine at residue number 50 (K*). The hypusine region is underlined.

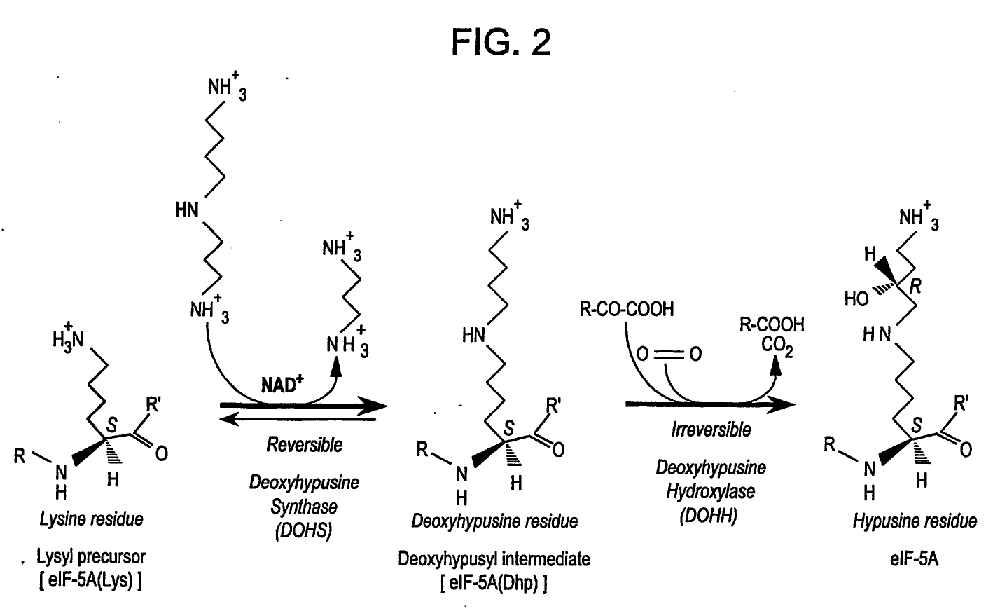

FIG. 2: Synthesis of the Hypusine Residue Defining Mature eIF-5A, Essential for its Biological Function.

The genetically not encoded hypusine residue within eif-5A is generated by a two-step post-translational modification of a genetically encoded lysine side chain. Spermidine is stoichiometrically consumed in the first, molecular oxygen in the second step. Thus, eIF-5A exists in three biosynthetic forms, two of them half-products (lysine precursor; deoxyhypusine intermediate) and one of them the hypusine-containing, ie. mature eIF-5A.

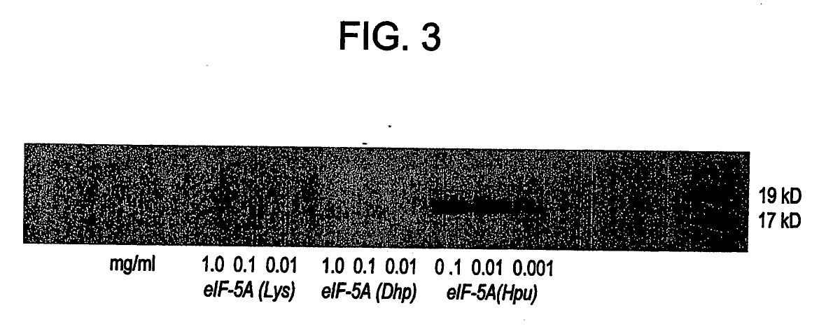

FIG. 3: Specificity of the Ligand NIH-353: Interaction with Only the Mature Variety Among the Three Biosynthetic Forms of eIF-5A.

Western blot of the lysine precursor [eIF-5A(Lys)], the deoxyhypusine intermediate [eIF-5A (Dhp)], and of the hypusine-containing mature eIF-5A [eIF-5A (Hpu)], each shown at decreasing concentrations of purified protein. NIH-353 does not interact with the lysine precursor even at 1 mg/ml, and interacts with the deoxyhypusine intermediate only marginally at concentrations that must exceed 100 ng/ml. By contrast, NIH-353 avidly binds to mature eIF-5A even at concentrations as low as 1 μg/ml. Molecular weight markers are indicated in kD.

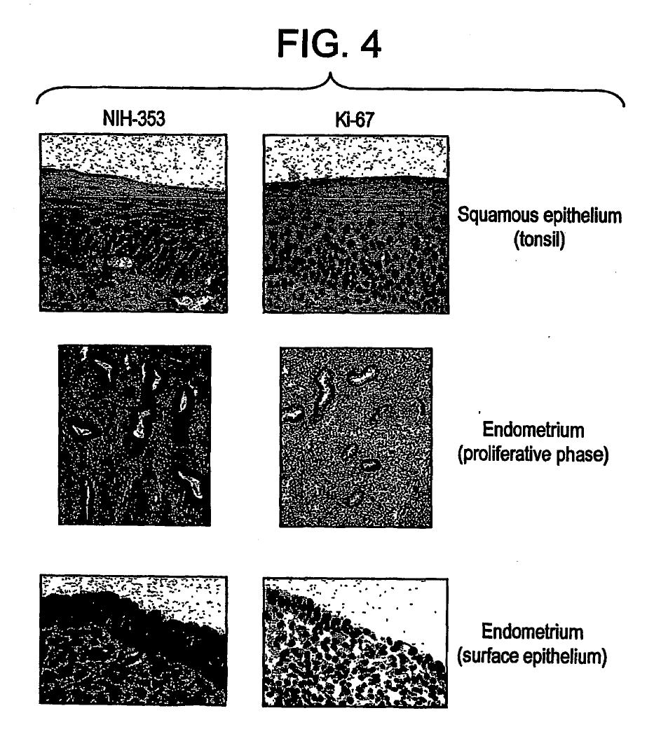

FIG. 4: Selectivity of the Ligand NIH-353: Labeling of Cells Preferentially in the Proliferative Zones of Human Tissues.

Typical results obtained in human tissues are exemplified by squamous epithelium, endometrium, and endometrial surface epithelium. Counterstaining of slides was performed with hematoxylin-eosin. Staining obtained with Ki-67, a standard antibody widely used in pathology to detect proliferating cells in human tissue samples, is shown for comparison. NIH-353 produces a signal that localizes to the cytoplasm, Ki-67 a signal that localizes to the nucleus. Thus, NIH-353 does not stain the nuclei, and Ki-67 does not stain the cytoplasm, of proliferating cells. In the squamous epithelium, NIH-353 and Ki-67 label the proliferating cells in the basal layers. In endometrium, NIH-353 and Ki-67 label the proliferating endometrial glands. In endometrial surface epithelium, NIH-353 and Ki-67 label the proliferating cells but not the underlying stroma. Visual clarity of the immunohistological label, for instance, in comparison to the counterstain is in part lost upon black- and white-reproduction of the image.

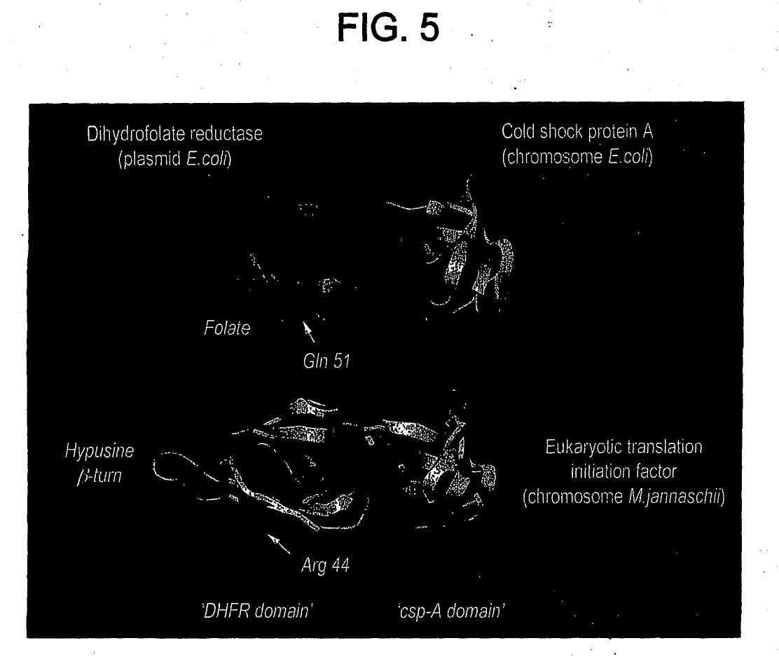

FIG. 5: Crystal Structure Comparison

Alignment of the crystal structure of the N-terminal part of eIF-5A of M. jannaschii (PDB# 1EIF) with the crystal structure of plasmid-encoded dihydrofolate reductase (DHFR) of E. coli (PDB# 1vie), using the Dali algorithm (Z score=4.4). Also shown is the alignment with cold shock protein A (csp-A) of E. coli.

FIG. 6: Sequence Comparison

Sequence alignment between human dihydrofolate reductase (DHFR) (Acc.# XM_{—}165390) and the human eIF-5As (I: Acc.# NP_{—}001961; II: Acc.# NP_{—}065123). Residues involved in the binding of the redox co-factor NADPH and of the molecular domains of folate (pterin, pABA, and glutamate) are indicated. The table summarizes the percent identity and similarity between the human DHFR and the human eIF-5As.

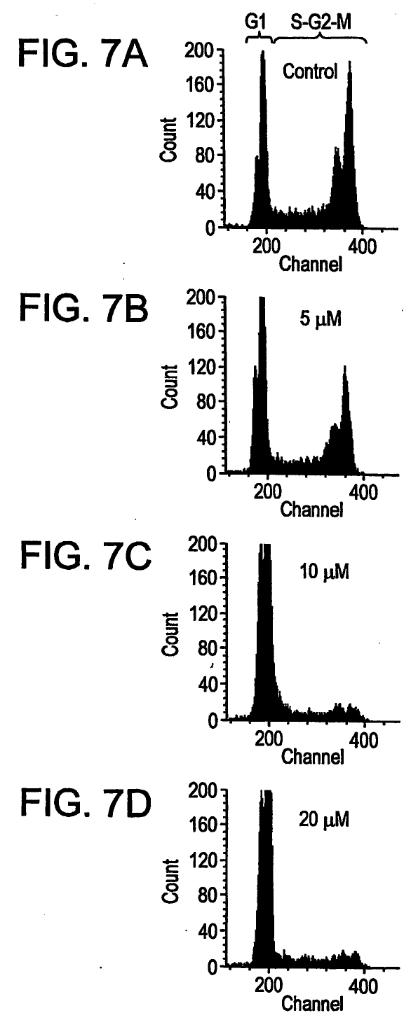

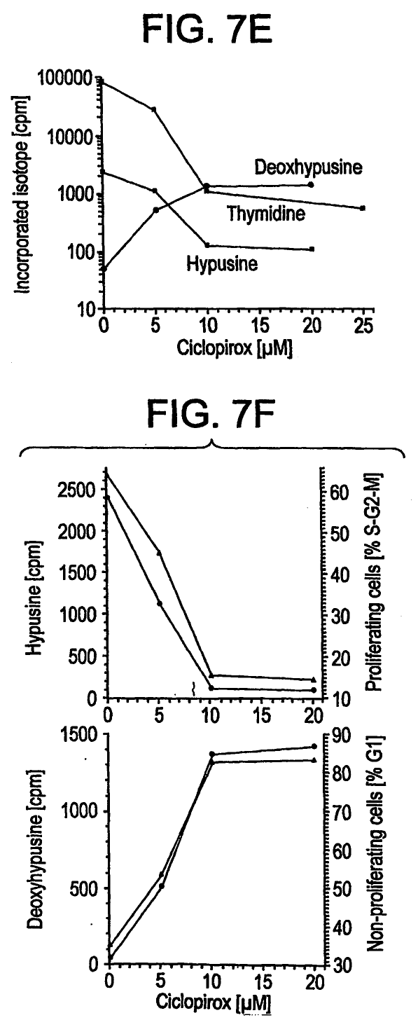

FIG. 7: Relation Between Hypusine Formation and Cell Proliferation in a Representative Human Cell Line

Effect of the fungicide ciclopirox on cell cycle progression, thymidine incorporation, and cellular deoxyhypusine hydroxylase activity, as also shown in PCT/US02/26909. Results are for the human cervical cancer cell line SiHa. Note the effect of increasing concentrations of ciclopirox on the proliferative S/G2/M compartment of the cell cycle (Plate A-D), on the incorporation of thymidine relative to the synthesis of hypusine and the accumulation of its precursor deoxyhypusine (Panel E), and on the relation between hypusine and deoxyhypusine relative to the percentage of cells proliferating (S/G2/M compartment) and non proliferating (G1), respectively (Panel F).

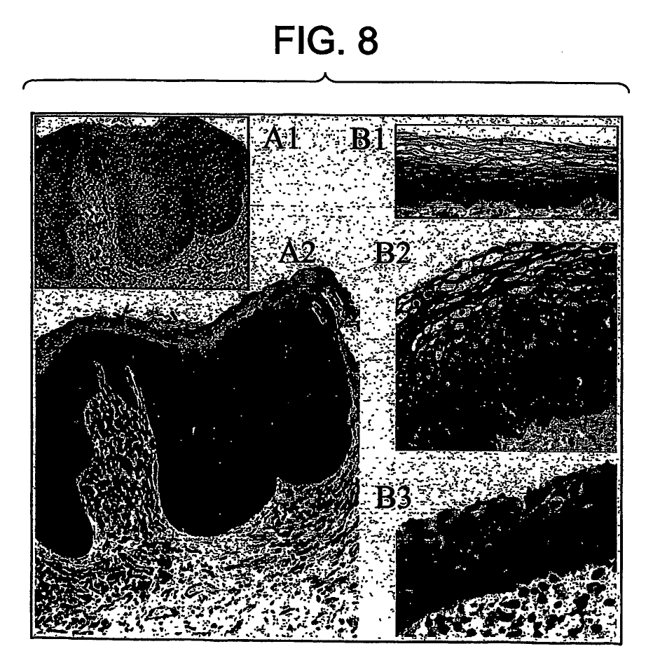

FIG. 8: Detection of Proliferative Cells in Normal and Neoplastic Epithelium.

A: Intraepithelial neoplasia of the vulva, grade III (A1: Staining with Ki-67; A2: Staining with NIH353). B: Cervical epithelium and intraepithelial neoplasia (B1: Normal cervical epithelium, with staining confined to only the physiologically proliferating cells in the basal/parabasal layer; B2: Low grade and B3: High grade intraepithelial neoplasia, with staining throughout the entire squamous epithelium). Visual clarity of the immunohistological label, for instance, in comparison to the counterstain is in part lost upon black- and white-reproduction of the image.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

### Definitions

The terms used herein have the meanings recognized and known to those of skill in the art, however, for convenience and completeness, particular terms and their meanings are set forth below.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

The term “antibody” as used herein includes intact molecules as well as fragments thereof, such as Fab and F(ab′)₂, which are capable of binding the epitopic determinant. Antibodies that bind the proteins of the present invention can be prepared using intact polypeptides or fragments containing small peptides of interest as the immunizing antigen attached to a carrier molecule. Commonly used carriers that are chemically coupled to peptides include bovine or chicken serum albumin, thyroglobulin, and other carriers known to those skilled in the art. The coupled peptide is then used to immunize the animal (e.g, a mouse, rat or rabbit). The antibody may be a “chimeric antibody”, which refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a human or a humanized antibody. The antibody may be a single chain antibody. (See, e.g., Curiel et al., U.S. Pat. No. 5,910,486 and U.S. Pat. No. 6,028,059). The antibody may be prepared in, but not limited to, mice, rats, rabbits, goats, sheep, swine, dogs, cats, or horses.

The term “antibody homologue” as used herein refers to whole immunoglobulin molecules, immunologically active portions or fragments thereof and recombinant forms of immunoglobulin molecules, or fragments thereof, that contain an antigen binding site which specifically binds (immunoreacts with) an antigen (e.g., cellular protein). Additionally, the term antibody homologue is intended to encompass non-antibody molecules that mimic the antigen binding specificity of a particular antibody. Such agents are referred to herein as “antibody mimetic agents”.

Structurally, the simplest naturally occurring antibody (e.g., IgG) comprises four polypeptide chains, two heavy (H) chains and two light (L) chains inter-connected by disulfide bonds. It has been shown that the antigen-binding function of an antibody can be performed by fragments of a naturally-occurring antibody. Thus, these antigen-binding fragments are intended to be encompassed by the term “antibody homologue”. Examples of binding fragments include (i) a Fab fragment consisting of the VL, VH, CL and CH1 regions; (ii) a Fd fragment consisting of the VH and CH1 regions; (iii) a Fv fragment consisting of the VL and VH regions of a single arm of an antibody, (iv) a dAb fragment, which consists of a VH region; (v) an isolated complimentarity determining region (CDR); and (vi) a F(ab′)₂ fragment, a bivalent fragment comprising two Fab fragments linked by a disulfide bridge at the hinge region.

Furthermore, although the two regions of the Fv fragment are coded for by separate genes, a synthetic linker can be made that enables them to be made as a single chain protein (referred to herein as single chain antibody or a single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antibody homologue”. Other forms of recombinant antibodies, such as chimeric, humanized and bispecific antibodies are also within the scope of the invention.

As used herein, the term “single-chain antibody” refers to a polypeptide comprising a V_{H} region and a V_{L} region in polypeptide linkage, generally linked via a spacer peptide (e.g., [Gly-Gly-Gly-Gly-Ser]ₓ), and which may comprise additional amino acid sequences at the amino- and/or carboxy-termini. For example, a single-chain antibody may comprise a tether segment for linking to the encoding polynucleotide. As an example, a scFv (single chain fragment variable) is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp. 361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope).

The term “antibody combining site”, as used herein refers to that structural portion of an antibody molecule comprised of a heavy and light chain variable and hypervariable regions that specifically binds (immunoreacts with) antigen.

The terms “bind”, “immunoreact” or “reactive with” in its various forms is used herein to refer to an interaction between an antigenic determinant-containing molecule (i.e., antigen) and a molecule containing an antibody combining site, such as a whole antibody molecule or a portion thereof, or recombinant antibody molecule (i.e., antibody homologue).

The term “monoclonal antibody” or “monoclonal antibody composition”, as used herein, refers to a population of antibody molecules that contain only one species of an antigen binding site capable of immunoreacting with a particular epitope of an antigen. A monoclonal antibody composition thus typically displays a single binding affinity for a particular antigen with which it immunoreacts.

The term “immunogen” is used herein to describe a composition typically containing a peptide or protein as an active ingredient (i.e., antigen) used for the preparation of antibodies against the peptide or protein.

“Analog” as used herein, refers to a chemical compound, a nucleotide, a protein, or a polypeptide that possesses similar or identical activity or function(s) as the chemical compounds, nucleotides, proteins or polypeptides having the desired activity and therapeutic effect of the present invention (eg. to inhibit cellular proliferation and to sensitize for, or potentiate chemotherapy or radiation therapy for treatment of mammals having cancer or hyperproliferative disorders), but need not necessarily comprise a sequence that is similar or identical to the sequence of the preferred embodiment, or possess a structure that is similar or identical to the agents of the present invention. As used herein, a nucleic acid or nucleotide sequence, or an amino acid sequence of a protein or polypeptide is “similar” to that of a nucleic acid, nucleotide or protein or polypeptide having the desired activity if it satisfies at least one of the following criteria: (a) the nucleic acid, nucleotide, protein or polypeptide has a sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleic acid, nucleotide, protein or polypeptide sequences having the desired activity as described herein (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the AAPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the polypeptides of the present invention having the desired therapeutic effect. As used herein, a polypeptide with “similar structure” to that of the preferred embodiments of the invention refers to a polypeptide that has a similar secondary, tertiary or quarternary structure as that of the preferred embodiment. The structure of a polypeptide can be determined by methods known to those skilled in the art, including but not limited to, X-ray crystallography, nuclear magnetic resonance, and crystallographic electron microscopy.

“Derivative” refers to either a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide. It may also refer to chemically synthesized organic molecules that are functionally equivalent to the active parent compound, but may be structurally different. It may also refer to chemically similar compounds which have been chemically altered to increase bioavailability, absorption, or to decrease toxicity.

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

As used herein “ligand” refers to a molecule, such as a peptide, which may be, but is not limited to, an antibody or fragment thereof, that is recognized by a particular receptor. As one of skill in the art will recognize, a molecule (or macromolecular complex) can be both a receptor and a ligand. In general, the binding partner having a smaller molecular weight is referred to as the ligand and the binding partner having a greater molecular weight is referred to as a receptor.

A “therapeutically effective amount” is an amount sufficient to decrease or prevent the symptoms associated with the cancer or hyperproliferative disorders or other related conditions contemplated for therapy with the compositions of the present invention.

“Treatment” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

“Combination therapy” refers to the use of the agents of the present invention with other active agents or treatment modalities, in the manner of the present invention for treatment of cancers or hyperproliferative disorders. These other agents or treatments may include drugs such as other anti-cancer drugs such as those that are standardly used to treat various cancers, radiation therapy, anti-viral drugs, corticosteroids, non-steroidal anti-inflammatory compounds, other agents useful in treating or alleviating pain, growth factors, cytokines, or colony stimulating factors. The combined use of the agents of the present invention with these other therapies or treatment modalities may be concurrent, or the two treatments may be divided up such that the agent of the present invention may be given prior to or after the other therapy or treatment modality.

“Local administration” means direct administration by a non-systemic route at or in the vicinity of the site of an affliction, disorder, or perceived pain.

“Slow release formulation” refers to a formulation designed to release a therapeutically effective amount of a drug or other active agent such as a polypeptide or a synthetic compound over an extended period of time, with the result being a reduction in the number of treatments necessary to achieve the desired therapeutic effect. In the matter of the present invention, a slow release formulation would decrease the number of treatments necessary to achieve the desired effect in terms of inhibiting cellular proliferation and decreasing the tumor burden or metastatic potential of a cancer or hyperprolilferative disorder.

The term “hyperproliferative disorders” refers to diseases that result from the abnormal growth of cells. These can include cancers and their precursors, as well as inflammatory states, for example, inflammations of blood vessels; and conditions involving unwanted proliferation of reactive immunocompetent cells, such as in rheumatoid arthritis, or multiple sclerosis; or abnormal proliferation of cells in other tissues of the human body, exemplified by psoriasis.

The term “immunosuppression” as defined herein refers to a situation that occurs when lymphocytes, which may include either T and/or B cell clones, or other cells of the immune system, such as macrophages or antigen presenting dendritic cells, are depleted in number or suppressed in their reactivity, expansion or differentiation. It may arise from activation of specific or non-specific T suppressor lymphocytes of either T or B cell clones or by drugs that have generalized effects on most or all T or B lymphocytes, as well as other cells in the immune system.

A “homologue” refers to polypeptides having the same or conserved residues at a corresponding position in their primary, secondary or tertiary structure. The term also extends to two or more nucleotide sequences encoding the homologous polypeptides.

A “vector” is a DNA molecule, capable of replication in a host organism, into which a gene is inserted to construct a recombinant DNA molecule.

“Biological activity of the hypusine region of hypusine-containing, or mature, eIF-5A”, as used herein, refers to the function of said protein in the metabolism of specific mRNAs, and their translatability into the corresponding proteins. In particular, the translatability of such mRNAs is required for the production of proteins that are essential for DNA replication and cellular proliferation. The general mechanism is translational control of gene expression.

“Surrogate biomarker” as used herein, refers to a highly specific molecule, the existence and levels of which are causally connected to a complex biological process, and reliably captures the state of said process. Furthermore, a surrogate biomarker, to be of practical importance, must be present in samples that can be obtained from individuals without endangering their physical integrity or well-being, preferentially from biological fluids such as blood, urine, saliva or tears.

For the purpose of this invention, the term “eIF-5A precursor” designates the immediate product of the eIF-5A gene, ie. the lysine form of the protein for which the abbreviation “eIF-5A (Lys)” has been introduced by Park and Wolff in the literature.

Similarly, as used herein, the term ‘eIF-5A intermediate”, designates the product of the first post-translational modification of eIF-5A (Lys), ie. the deoxyhypusine form of the protein for which the abbreviation “eIF-5A (Dhp)” has been introduced by Park and Wolff in the literature.

The term “mature eIF-5A”, as used herein, designates the product of the second post-translational modification, ie. the hypusine-containing form of the protein for which the abbreviation “eIF-5A (Hpu)” has been introduced by Park and Wolff in the literature. This “mature” form of eIF-5A mediates the biological activity of the eIF-5A genes, as defined above.

The “class of hypusine antigens”, as used herein, consists of mature eIF-5A, its hypusine-containing peptide fragments and degradation product, as well as, free hypusine.

Enzymes mediating post-translational modification of specific, peptide bound amino acid residues, such as the hydroxylases of collagenous proteins, are conventionally designated by the ending “yl”, attached to the identifier of the modified residue. This convention reemphasizes that such enzymes do not modify a residue if it is not within a peptide linkage. For example, the enzyme that hydroxylates certain proline residues in collagenous proteins are termed “prolyl” hydroxylases, but do not act on free proline; only proline hydroxylase does. However, this convention has not been extended to eIF-5A investigations, consistent with the predominant usage in this application, the terms deoxyhypusine synthase (DOHS), but not deoxyhypusyl synthase, and the term deoxyhypusine hydroxylase (DOHH), but not the term deoxyhypusyl hydroxylase are used. Both enzymes only modify peptide bound residues.

### General Description of the Invention

The eukaryotic translation initiation factor 5A exists in two genetically distinct variants, 1 and 2, which both contain a single hypusine residue, formed by enzymatic hydroxylation within a collagen motif of the sequence -Gly-X-Y-Gly- [FIG. 1]. Hypusine is not genetically encoded. The residue derives from a genetically encoded lysine moiety, after butylamine transfer utilizing spermidine and hydroxylation utilizing atmospheric oxygen [FIG. 2]. In culture, reversible suppression of hypusine formation correlates with reversible arrest in the late G1 phase of the cell cycle, immediately before the initiation of DNA replication (Hanauske-Abel, H. M., et al., (1994) Biochim. Biophys. Acta 1221, 115-124).

Inhibitors of deoxyhypusyl hydroxylase (DOHH), the hydroxylating enzyme, typically cause arrest at the immediate G1/S boundary of the cell cycle jointly with the disappearance from polysomes of a unique subset of cellular mRNA's termed hymns (hypusine-dependent messenger nucleic acids) (Hanauske-Abel, H. M., et al. (1995) FEBS Lett. 366, 92-98.). Reactivation of DOHH causes rapid reappearance of hymns at polysomes and subsequent, highly synchronized entry of cells into S phase (Hanauske-Abel, H. M., et al. (1995) FEBS Lett. 366, 92-98.). The hymns, though encoding very diverse cell cycle-relevant proteins, may share common nucleotide motifs, termed JSBs, in their untranslated 3′ and 5′ regions. Recent structural analyses of hypusine-containing eIF-5A indicate that its C-terminal part folds like the cold-shock protein A of E. coli, which prevents mRNA duplex formation at low temperatures, and that its N-terminal part contains motifs II, III, IV, and V of ATP-utilizing mRNA helicases, required for unwinding of mRNA duplexes (H M Hanauske-Abel, et al. (2002), FASEB J. 16, A549). DOHH activity is also required for the intracellular multiplication of retroviruses, in particular human immunodeficiency virus (HIV) whose genome encodes Rev, a protein reported to interact with hypusine-containing eIF-5A under certain conditions (Andrus, L. et al. (1998), Biochem. Pharmacol. 55: 1807-1808). Inhibition of DOHH activity suppresses the formation of infectious HIV virions by removing from the host cell polysomes the retroviral mRNAs encoding the capsid proteins.

Therefore, the presence or absence, respectively, of hypusine in eIF-5A, mediated by activity or inactivity, respectively, of DOHH, correlates with the initiation or the cessation, respectively, of proliferation of cells as well as the initiation or the cessation, respectively, of multiplication of infectious HIV particles. Cognizant of the biological importance of hypusine and the hypusine region, the Applicants of the present invention hypothesized that ligands that bind to hypusine-containing eIF-5A, and in particular its hypusine region, would identify those cells in tissues that are in the process of proliferation, i.e. initiating or undergoing replication of their DNA. The identification of such ligands has multiple applications in conditions that involve cell proliferation.

In particular, the Applicants of the present invention have generated antibodies against the structure of bioactive hypusine-containing eIF-5A isolated from human red blood cells and have performed experiments, the data of which is included herein, to support a role for such ligand in diagnostic and therapeutic applications.

Surprisingly, although of polyclonal origin, one particular antibody that was generated was entirely non-reactive with the protein as encoded by the human eIF-5A genes (‘lysine precursor’), i.e. the form lacking hypusine entirely and displaying a lysine side chain instead. The protein representing the half-product formed during post-translational modification (‘deoxyhypusine intermediate’) was marginally reactive with the polyclonal antibody. In contradistinction, the protein representing the final product formed by post-translational modification, i.e. the mature eIF-5A, was highly reactive with antibody designated NIH-353. The specificity of NIH-353 for eIF-5A if it contains hypusine makes this antibody a principal tool for the identification of natural and man-made molecules that are able to bind to, or otherwise interact with, the hypusine region of mature eIF-5A. Such identification may employ, and rely on, NIH-353, fragments or derivatives thereof, in a number of techniques, exemplified by competitive assays.

When used as a reagent in routine immunohistochemistry procedures, the NIH-353 antibody did not generate any distinctive staining of human tissues. Surprisingly, however, standard antigen retrieval methods (MacIntyre, N. (200), British Journal of Biomedical Science, 58:190-196) performed on tissue slides did render the same NIH-353 antibody highly reactive. Only the proliferating cells in antigen retrieval treated tissue sections were labeled by NIH-353 (comp. FIG. 4). This is entirely consistent with the extensive in vitro data on the essential role that the hypusine region of mature eIF-5A plays in cell proliferation.

To obtain additional data on the interaction of hypusine-containing eIF-5A with specific mRNAs essential for cell cycle control, further database analysis revealed amino acid sequence homology with the crystal of dihydrofolate reductase (DHFR). Folate, which is a vitamin required for metabolism of one-carbon units and essential for the biosynthesis of DNA and RNA building blocks, is also known to be involved in the translational control of gene expression. For instance, whereas the levels of the mRNA encoding dihydrofolate reductase or of the mRNA encoding the folate receptor alpha do not change in response to alterations of folate levels, the translation of these mRNAs occur in a manner that is sensitive to the concentration of folate or folate analogs and antagonists (Ercikan-Abali E A et al. (1997), Biochemistry 36, 12317-12322; Tai N, et al. (2002), Nucleic Acid Res. 15: 4481-4488; Zhu W Y, et al. (2001), J Cell Biochem 81, 205-219). Therefore, folate is a small molecular modifier of translational control of gene expression.

The Applicants of the present invention noted that the eIF-5As display significant structural homologies with at least one of the proteins whose translational efficiency is affected by folate/antifolate levels: the dihydrofolate reductases (DHFRs). Dihydrofolate reductase (E.C.1.5.1.3; 5, 6, 7, 8-tetrahydrofolate:NADP+oxoreductase) is required to maintain the intracellular pool of reduced folates in rapidly dividing cells. Inhibitors of this enzyme have proven effective in antineoplastic, antiparasitic, antimicrobial, and immunosuppressive chemotherapy. Methotrexate, an analog that preserves the basic folate (pteroylglutamate) structure, and is representative of the entire class of anti-folates, potently inhibits DHFR from mammalian and bacterial sources, but requires transport by a folate-specific membrane carrier found only on mammalian cells, and is therefore primarily useful as an antineoplastic agent.

Recent structural analyses of eIF-5A indicate that its C-terminal part folds like the cold-shock protein A of E. coli, (Peat T S, et al. (1998), Structure 6, 1207-1214), and that its most N-terminal part contains motifs II, III, IV, and V of ATP-utilizing mRNA helicases, required for unwinding of mRNA duplexes (H M Hanauske-Abel, et al. (2002), FASEB J. 16, A549). Using the spatial coordinates of only the N-terminal part of eIF-5A of M. jannaschii (PDB# 1EIF), Applicants noted a significant homology with the crystal structure of plasmid-encoded DHFR of E. coli (PDB# 1vie), using the Dali algorithm (Z score=4.4), see FIG. 5.

Similarly, optimized sequence alignment between human DHFR (Acc.# XM_{—}165390) and the human eIF-5As (1: Acc.# NP_{—}001961; 2: Acc.# NP_{—}065123) revealed 37% identify/similarity with eIF-5A-I and 35% identify/similarity with eIF-5A-II. The N-terminal region of the human eIF-5As displays several isolocated residues that in DHFR participate in binding of folate and the antifolate methotrexate (e.g. Ile⁷, Pro⁶¹, Arg⁷⁰), and of NADPH (e.g. Gly²⁰, Lys⁵⁴, Gly¹¹⁷, Ser¹¹⁸). Distinct sequence differences affecting residues involved in catalytic efficiency, such as the E30Q isolocation, suggest the eIF-5As display limited if any DHFR activity, see FIG. 6.

Applicants hypothesized that in eIF-5A, the DHFR-like structural motifs function in rendering the translational control of hymns sensitive to levels of folate and its analogs. The discovery within eIF-5As of substructures that are a prerequisite for binding of folate and of folate derivatives identifies the eIF-5As as potential targets for these small molecule ligands, and points to multiple applications in conditions that involve cell proliferation.

Applicants noted marked similarities between eIF-5A and dihydrofolate reductase at the level of their primary as well as their tertiary structures, involving in particular motifs that in the enzyme enable the binding of folate and its derivatives. eIF-5A and dihydrofolate reductase are both known to control translational efficiancy of specific mRNAs, the latter in a manner controlled by the level of folate/antifolate.

As demonstrated herein, the outlined results represent information directly enabling the use of folate and its derivatives, and the discovery and development of novel folate analogs, that modulate the translational control of gene expression executed by eIF-5A.

### Development of Folate Analogs that Bind the eIF-5A Molecule

Thus, the invention provides for a ligand specific for the folate-binding REGION of eukaryotic translation initiation factor 5A. This folate-binding region comprises at least one residue motif common to eIF-5A and to the bacterial and human dihydrofolate reductases as shown in FIG. 6. In a preferred embodiment, the ligand is selected from the group consisting of an analog of folate, derivatives thereof and fragments thereof, which specifically bind to an eIF-5A molecule only if the eIF-5A contains a folate-binding region. Preferred embodiments include, but are not limited to, anti-folates like methotrexate, aminopterine, trimetrexate, lomextrexol, pemetrexed, and newer compounds such as the pyrrolo[2,3-d]pyrimidines, eg. TNP-351, or the cyclopenta[d]pyrimidine derivatives.

A further aspect of the invention provides for a method for identifying folate derivatives that are inhibitors of proliferation, yet it is desirable that these inhibitors do not inhibit folate-dependent enzymes. A preferred embodiment provides for placing the folate derivatives under investigation in contact with an eIF-5A molecule containing a folate-binding region, and measuring the extent, if any, to which the folate derivatives specifically bind to the eIF-5A molecule. In a further preferred embodiment, the folate derivatives under investigation are placed in contact with the eIF-5A molecule containing a folate-binding region, and with the ligand, and measuring the extent to which the folate derivatives successfully compete with the ligand for binding with the eIF-5A molecule.

A further aspect of the invention provides for a method for inhibiting in a cell the biological activity of the folate-binding region of eIF-5A, the folate binding region comprising residue motifs as set forth in FIG. 6. This method provides for introducing into a cell a low-molecular weight molecule that binds to the folate-binding region of eIF-5A, and to thereby inhibit the biological activity of eIF-5A required for cell proliferation. The biological activity of eIF-5A may involve cellular proliferation. It may also involve replication of HIV.

The term “neoplastic disease” as used herein refers to an abnormal state or condition characterized by rapidly proliferating cell growth or neoplasm. Based upon standard laboratory experimental techniques and procedures well known and appreciated by those skilled in the art, as well as upon comparisons with compounds of known usefulness, the agents described herein are useful in the treatment of patients suffering from those neoplastic diseases which generally are or can be treated with folates and antifolates such as methotrexate, aminopterin, 5,10-dideazafolate and leucovorin. Such neoplastic diseases may include: leukemias, including but not limited to acute lymphoblastic, chronic lymphocytic, acute myeloblastic and chronic myelocytic; lymphomas; carcinomas, including but not limited to those of the cervix, esophagus, stomach, small intestine, colon and lungs; sarcomas, including but not limited to osteosarcoma, liposarcoma, and hemangiosarcoma; melanomas, including amelanotic and melanotic; and mixed types of neoplasms such as, for example, carcinosarcoma. Of course, one skilled in the art will recognize that not every compound identified by the methods described herein will be effective against each of the neoplastic disease states, and that selection of the most appropriate compound is within the ability of one of ordinary skill in the art and will depend on a variety of factors including assessment of results obtained in standard animal tumor models. In general the compounds that may be identified by the methods described herein are useful in the treatment of those neoplastic diseases currently treated with folates and antifolates.

The term “antineoplastic effect” and the term “treating a cancer or neoplastic disease” refers to an effect of controlling the growth or proliferation of the neoplasm or in prolonging the survivability of the patient beyond that expected in the absence of such treatment. The growth or proliferation of a neoplasm is controlled by slowing, interrupting, arresting or stopping its formation, and once formed, its growth, proliferation or its metastases. The term “treating a neoplastic disease” therefore does not necessarily indicate a total elimination of the neoplastic disease. It is believed that prolonging survival, or improving quality of life by decreasing symptomatology, is a significant advantageous effect in and of itself.

Methotrexate and other folate and antifolate agents have also been employed in the treastment of psoriasis, a disease characterized by an increased rate of epidermal cell proliferation. The compounds identified by the methods described herein are expected to be valuable new agents in the treatment of psoriasis and similar conditions. The antineoplastic dosage may be the same as the antipsoriasis dosage, except that when the compounds so identified are used in the treatment of psoriasis, topical application would be preferred.

The compounds can be administered alone or in the form of a pharmaceutical composition in combination with pharmaceutically acceptable carriers or excipients, the proportion and nature of which are determined by the solubility and chemical properties of the compound selected, the chosen route of administration, and standard pharmaceutical practice. The compounds identified, while effective themselves, may be formulated and administered in the form of their pharmaceutically acceptable acid addition salts for purposes of stability, convenience of crystallization, increased solubility and the like.

In certain embodiments, a compound of the invention is administered to a patient, preferably a mammal, more preferably a human, as a treatment against cancer, hyperproliferative disorders or a viral infection, in particular, HIV. In one embodiment, a compound of the invention is administered as a therapeutic measure to a patient. According to this embodiment, the patient can have a genetic or a non-genetic predisposition to cancer, hyperproliferative disorders, or a viral infection.

In certain embodiments of the present invention, an agent/compound identified by the methods of the present invention can be used in combination therapy with at least one other therapeutic agent. The compound of the invention and the therapeutic agent can act additively or, more preferably, synergistically. In a preferred embodiment, a composition comprising a compound of the invention is administered concurrently with the administration of another therapeutic agent, which can be part of the same composition as or in a different composition from that comprising the compound of the invention. In another embodiment, a composition comprising a compound of the invention is administered prior or subsequent to administration of another therapeutic agent. As many of the disorders for which the compounds of the invention are useful in treating are chronic, in one embodiment combination therapy involves alternating between administering a composition comprising a compound of the invention and a composition comprising another therapeutic agent, e.g., to minimize the toxicity associated with a particular drug. The duration of administration of the compound of the invention or therapeutic agent can be, e.g., one month, three months, six months, a year, or for more extended periods, or on an alternate or intermittent schedule. In certain embodiments, when a compound of the invention is administered concurrently with another therapeutic agent that potentially produces adverse side effects including, but not limited to, toxicity, the therapeutic agent can advantageously be administered at a dose that falls below the threshold at which the adverse side is elicited.

The therapeutic agent can be another anti-cancer agent. Useful anti-cancer agents include, but are not limited to, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, radiation, alkylating agents including nitrogen mustard such as cyclophosphamide, Ifosfamide, trofosfamide, Chlorambucil, nitrosoureas such as carmustine (BCNU), and Lomustine (CCNU), alkylsulphonates such as busulfan, and Treosulfan, triazenes such as Dacarbazine, platinum containing compounds such as Cisplatin and carboplatin, plant alkaloids including vinca alkaloids, vincristine, Vinblastine, Vindesine, and Vinorelbine, taxoids including paclitaxel, and Docetaxol, DNA topoisomerase inhibitors including Epipodophyllins such as etoposide, Teniposide, Topotecan, 9-aminocamptothecin, mytomycins such as mytomycin C, anti-metabolites, including other known anti-folates such as DHFR inhibitors, Trimetrexate, IMP dehydrogenase inhibitors including mycophenolic acid, Tiazofurin, Ribavirin, ribonucleotide reductase inhibitors such as hydroxyurea, deferoxamine, pyrimidine analogs including uracil analogs 5-Fluorouracil, Floxuridine, Doxifluridine, and Ratitrexed, cytosine analogs such as cytarabine, cytosine arabinoside, and fludarabine, purine analogs such as mercaptopurine, thioguanine, hormonal therapies including receptor antagonists, the anti-estrogens Tamoxifen, Raloxifene and megestrol, LHRH agonists, Leuprolide acetate, anti-androgens such as flutamide, and bicalutamide, retinoids/deltoids, Vitamin D3 analogs, photodyamic therapies including vertoporfin, Phthalocyanine, cytokines including Interferons, tumor necrosis factor, as well as other compounds having anti-tumor activity including Isoprenylation inhibitors such as Lovastatin, Dopaminergic neurotoxins such as 1-methyl-4-phenylpyridinium ion, Cell cycle inhibitors such as staurosporine, Actinomycins, Bleomycins, anthracyclines such as daunorubicin, doxorubicin, Idarubicin, Epirubicin, Pirarubicin, Zorubicin, and Mitoxantrone; or similar agents in this group of drugs.

The therapeutic agent can be an anti-inflammatory agent or an analgesic agent. Useful anti-inflammatory and analgesic agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, colchicine, allopurinol, probenecid, sulfinpyrazone and benzbromarone or similar agents in this group of drugs.

The therapeutic agent can be an antiviral agent. Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons or similar agents in this group of drugs.

### Antibodies Specific for the Hypusine Region of eIF-5A

Furthermore, the invention provides antibodies specific for hypusine-containing eIF-5A from human red blood cells. Mature eIF-5A was used to immunize rabbits to generate antibodies to mature eIF-5A using standard methodologies. One antibody, designated ‘NIH 353’, binds the hypusinated (mature) form of eIF-5A. Specifically, this ‘NIH 353 binds the hypusine-containing region of eIF-5A; however, ‘NIH 353’ does not bind with the same affinity to the forms containing lysine or deoxyhypusine, the biochemical precursors of hypusine. Immunocytochemistry analysis revealed that proliferating cells readily bound ‘NIH 353’ antibody. Formalin-fixed, paraffin-embedded human tonsils were sectioned such that they contained two proliferative areas, the germinal centers of lymphoid follicles and the basal layer of the squamous epithelium. Following a well-known optimal antigen retrieval protocol involving microwave irradiation, the sections were contacted with ‘NIH 353’, and binding of ‘NIH 353’ to hypusine-containing eIF-5A was detected using standard streptavidin-biotin/horseradish peroxidase methodologies with diaminobenzidine as the chromogen and hematoxylin as the counterstain. The cytoplasm of cells only in the basal layer of the epithelium and in the germinal centers stained prominently. Nuclei remained unlabelled. For comparison, these same tissues were then reacted with antibodies against Ki-67 nuclear antigen, a known proliferative marker. Similar results were obtained when ‘NIH 353’ was reacted with endometrium.

Antibodies or antibody fragments of the invention, which are specific for mature eIF-5A may be natural or partially or wholly synthetically produced. All derivatives thereof which maintain specific eIF-5A binding ability are also included. The antibodies may be monoclonal or polyclonal and may be a member of any immunoglobulin class, including any of the human classes: IgG, IgM, IgA, IgD, and IgE. Derivatives of the IgG class, however, are preferred in the present invention.

Antibody fragments recognizing mature eIF-5A may be any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. The antibody fragment may be produced by any means. For instance, the antibody fragment may be enzymatically or chemically produced by fragmentation of an intact antibody or it may be recombinantly produced from a gene encoding the partial antibody sequence. As used herein, antibody also includes bispecific and chimeric antibodies.

Naturally produced monoclonal antibodies may be generated using classical cloning and cell fusion techniques. In general, mature eIF-5A is administered (e.g., intraperitoneal injection) to wild-type or inbred mice (e.g., BALB/c) or transgenic mice which produce desired antibodies, or rats, rabbits, chickens, sheep, goats, or other animal species which can produce native or human antibodies. The mature eIF-5A may be administered alone, or mixed with adjuvant. After the animal is boosted, for example, two or more times, the spleen or large lymph node, such as the popliteal in rat, is removed and splenocytes or lymphocytes are extracted and fused with myeloma cells using well-known processes, for example Kohler and Milstein [(1975) Nature 256:495-497] and Harlow and Lane [Antibodies: A Laboratory Manual (Cold Spring Harbor Laboratory, New York (1988)]. The resulting hybrid cells are then cloned in the conventional manner, e.g. using limiting dilution, and the resulting clones, which produce the desired monoclonal antibodies, and are cultured.

Alternatively, antibodies against mature eIF-5A are derived by a phage display method. Methods of producing phage display antibodies are well-known in the art, for example, Huse, et al. [(1989) Science 246(4935): 1275-81].

Methods for identification and production of polynucleotides conferring a desired phenotype and/or encoding a protein, for example, an antibody, having an advantageous predetermined property which is selectable are known in the art (See for example, U.S. Pat. No. 6,576,467). Thus, in order to overcome many of the limitations in producing and identifying high-affinity immunoglobulins through antigen-stimulated B cell development (i.e., immunization and subsequent determination of the binding characteristics of the antibodies made), various prokaryotic expression systems are available which can be manipulated to produce combinatorial antibody libraries. Thereafter, these libraries may be screened for high-affinity antibodies to specific antigens, for example hypusine-containing eIF-5A. Recent advances in the expression of antibodies in Escherichia coli and bacteriophage systems have raised the possibility that virtually any specificity can be obtained by either cloning antibody genes from characterized hybridomas or by de novo selection using antibody gene libraries (e.g., from Ig cDNA).

Combinatorial libraries of antibodies have been generated in bacteriophage lambda expression systems which may be screened as bacteriophage plaques or as colonies of lysogens (Huse et al. (1989) Science 246: 1275; Caton and Koprowski (1990) Proc. Natl. Acad. Sci. (U.S.A.) 87: 6450; Mullinax et al (1990) Proc. Natl. Acad. Sci. (U.S.A.) 87: 8095; Persson et al. (1991) Proc. Natl. Acad. Sci. (U.S.A.) 88: 2432). Various embodiments of bacteriophage antibody display libraries and lambda phage expression libraries have been described (Kang et al. (1991) Proc. Natl. Acad. Sci. (U.S.A.) 88: 4363; Clackson et al. (1991) Nature 352: 624; McCafferty et al. (1990) Nature 348: 552; Burton et al. (1991) Proc. Natl. Acad. Sci. (U.S.A.) 88: 10134; Hoogenboom et al. (1991) Nucleic Acids Res. 19: 4133; Chang et al. (1991) J. Immunol. 147: 3610; Breitling et al. (1991) Gene 104: 147; Marks et al. (1991) J. Mol. Biol. 222: 581; Barbas et al. (1992) Proc. Natl. Acad. Sci. (U.S.A.) 89: 4457; Hawkins and Winter (1992) J. Immunol. 22: 867; Marks et al. (1992) Biotechnology 10: 779; Marks et al. (1992) J. Biol. Chem. 267: 16007; Lowman et al (1991) Biochemistry 30: 10832; Lerner et al. (1992) Science 258: 1313, incorporated herein by reference). Typically, a bacteriophage antibody display library is screened with an antigen (e.g., polypeptide, carbohydrate, glycoprotein, nucleic acid) that is immobilized (e.g., by covalent linkage to a chromatography resin to enrich for reactive phage by affinity chromatography) and/or labeled (e.g., to screen plaque or colony lifts).

One particularly advantageous approach has been the use of so-called single-chain fragment variable (scFv) libraries (Marks et al. (1992) Biotechnology 10: 779; Winter G and Milstein C (1991) Nature 349: 293; Clackson et al. (1991) op.cit.; Marks et al. (1991) J. Mol. Biol. 222: 581; Chaudhary et al. (1990) Proc. Natl. Acad. Sci. (USA) 87: 1066; Chiswell et al. (1992) TIBTECH 10: 80; McCafferty et al. (1990) op.cit.; and Huston et al. (1988) Proc. Natl. Acad. Sci. (USA) 85: 5879). Various embodiments of scFv libraries displayed on bacteriophage coat proteins have been described.

Beginning in 1988, single-chain analogues of Fv fragments and their fusion proteins have been reliably generated by antibody engineering methods. The first step generally involves obtaining the genes encoding V_{H} and V_{L} REGIONs with desired binding properties; these V genes may be isolated from a specific hybridoma cell line, selected from a combinatorial V-gene library, or made by V gene synthesis. The single-chain Fv is formed by connecting the component V genes with an oligonucleotide that encodes an appropriately designed linker peptide, such as (Gly-Gly-Gly-Gly-Ser)₃ or equivalent linker peptide(s). The linker bridges the C-terminus of the first V region and N-terminus of the second, ordered as either V_{H}-linker-V_{L} or V_{L}-linker-V_{H}. In principle, the scFv binding site can faithfully replicate both the affinity and specificity of its parent antibody combining site.

Thus, scFv fragments are comprised of V_{H} and V_{L} REGIONs linked into a single polypeptide chain by a flexible linker peptide. After the scfv genes are assembled, they are cloned into a phagemid and expressed at the tip of the M13 phage (or similar filamentous bacteriophage) as fusion proteins with the bacteriophage pIII (gene 3) coat protein. Enriching for phage expressing an antibody of interest is accomplished by panning the recombinant phage displaying a population scFv for binding to a predetermined epitope (e.g., target antigen, receptor).

The linked polynucleotide of a library member provides the basis for replication of the library member after a screening or selection procedure, and also provides the basis for the determination, by nucleotide sequencing, of the identity of the displayed peptide sequence or V_{H} and V_{L} amino acid sequence. The displayed peptide(s) or single-chain antibody (e.g., scFv) and/or its V_{H} and V_{L} regions or their CDRs can be cloned and expressed in a suitable expression system. Often polynucleotides encoding the isolated V_{H} and V_{L} regions will be ligated to polynucleotides encoding constant regions (C_{H} and C_{L}) to form polynucleotides encoding complete antibodies (e.g., chimeric or fully-human), antibody fragments, and the like. Often polynucleotides encoding the isolated CDRs will be grafted into polynucleotides encoding a suitable variable region framework (and optionally constant regions) to form polynucleotides encoding complete antibodies (e.g., humanized or fully-human), antibody fragments, and the like. Antibodies can be used to isolate preparative quantities of the antigen by immunoaffinity chromatography. Various other uses of such antibodies are to diagnose and/or stage disease (e.g., neoplasia), and for therapeutic application to treat disease, such as for example: neoplasia, HIV infections and the like.

Various methods have been reported for increasing the combinatorial diversity of a scFv library to broaden the repertoire of binding species. The use of PCR (polymerase chain reaction) has permitted the variable regions to be rapidly cloned either from a specific hybridoma source or as a gene library from non-immunized cells, affording combinatorial diversity in the assortment of V_{H} and V_{L} cassettes which can be combined. Furthermore, the V_{H} and V_{L} cassettes can themselves be diversified, such as by random, pseudorandom, or directed mutagenesis. Typically, V_{H} and V_{L} cassettes are diversified in or near the complementarity-determining regions (CDRs), often the third CDR, CDR3. Enzymatic inverse PCR mutagenesis has been shown to be a simple and reliable method for constructing relatively large libraries of scFv site-directed mutants (Stemmer et al. (1993) Biotechniques 14: 256), as has error-prone PCR and chemical mutagenesis (Deng et al. (1994) J. Biol. Chem. 269: 9533). Riechmann et al. [Biochemistry 32: 8848; (1993)] showed semirational design of an antibody scFv fragment using site-directed randomization by degenerate oligonucleotide PCR and subsequent phage display of the resultant scFv mutants.

Selection of antibodies specific for mature eIF-5A is based on binding affinity to hypusine-containing eIF-5A and may be determined by various well-known immunoassays including, enzyme-linked immunosorbent, chemiluminescent, immunofluorescent, immunohistochemical, radioimmunoassay, and immunoprecipitation assays and the like which may be performed in vitro, in vivo or in situ. The standard techniques known in the art for immunoassays are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904.

### Use of Antibodies against Mature eIF-5A for Diagnostic Purposes

One aspect of the invention provides a method of using an antibody against mature eIF-5A to diagnose a hyperproliferative disorder in a subject. As hypusine-containing eIF-5A is essential for cell proliferation, it provides a general biomarker for hyperproliferative disorders in which cell growth is independent of normal regulatory mechanisms (e.g., loss of contact inhibition). This includes, but is not limited to, the abnormal growth of tumor cells, both benign and malignant, due to direct expression of an oncogene or as a result of oncogenic mutation in another gene, or as a result of aberrant cell cycle regulation. Thus, compositions and methods provided herein are particularly deemed useful for the diagnosis of hyperproliferative disorders including solid tumors such as skin, breast, brain, cervical carcinomas, testicular carcinomas, etc. More particularly, cancers that may be treated by the compositions and methods of the invention include, but are not limited to, Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma [squamous cell, undifferentiated small cell, undifferentiated large cell, adenocarcinoma], alveolar [bronchiolar] carcinoma, bronchial adenoma, sarcoma, lymphoma, chondromatous hamartoma, mesothelioma; Gastrointestinal: esophagus [squamous cell carcinoma, adenocarcinoma, leiomyosarcoma, lymphoma], stomach [carcinoma, lymphoma, leiomyosarcoma], pancreas [ductal adenocarcinoma, insulinoma, glucagonoma, gastrinoma, carcinoid tumors, VIPoma], small bowel [adenocarcinoma, lymphoma, carcinoid tumors, Kaposi's sarcoma, leiomyoma, hemangioma, lipoma, neurofibroma, fibroma], large bowel [adenocarcinoma, tubular adenoma, villous adenoma, hamartoma, leiomyoma]; Genitourinary tract: kidney [adenocarcinoma, Wilms tumor (nephroblastoma), lymphoma, leukemia], bladder and urethra [squamous cell carcinoma, transitional cell carcinoma, adenocarcinoma], prostate [adenocarcinoma, sarcoma], testis [seminoma, teratoma, embryonal carcinoma, teratocarcinoma, choriocarcinoma, sarcoma, Leydig cell tumor, fibroma, fibroadenoma, adenomatoid tumors, lipoma]; Liver: hepatocellular carcinoma, cholangiocarcinoma, hepatoblastoma, angiosarcoma, hepatocellular adenoma, hemangioma; Bone: osteogenic sarcoma [osteosarcoma], fibrosarcoma, malignant fibrous histiocytoma, chondrosarcoma, Ewing's sarcoma, malignant lymphoma [reticulum cell sarcoma], multiple myeloma, malignant giant cell tumor, chordoma, osteochondroma [osteocartilaginous exostoses], benign chondroma, chondroblastoma, chondromyxoid fibroma, osteoid osteoma and giant cell tumors; Nervous system: skull [osteoma, hemangioma, granuloma, xanthoma, Paget's disease of bone], meninges [meningioma, meningiosarcoma, gliomatosis], brain [astrocytoma, medulloblastoma, glioma, ependymoma, germinoma (pinealoma), glioblastoma multiforme, oligodendroglioma, schwannoma, retinoblastoma, congenital tumors], spinal cord [neurofibroma, meningioma, glioma, sarcoma]; Gynecological: uterus [endometrial carcinoma], cervix [cervical carcinoma, pre-invasive cervical dysplasia], ovaries [ovarian carcinoma (serous cystadenocarcinoma, mucinous cystadenocarcinoma, endometrioid carcinoma, clear cell adenocarcinoma, unclassified carcinoma), granulosa-theca cell tumors, Sertoli-Leydig cell tumors, dysgerminoma, malignant teratoma and other germ cell tumors], vulva [squamous cell carcinoma, intraepithelial carcinoma, adenocarcinoma, fibrosarcoma, melanoma], vagina [clear cell carcinoma, squamous cell carcinoma, sarcoma botryoides (embryonal rhabdomyosarcoma), fallopian tubes [carcinoma]; Hematologic: blood [myeloid leukemia (acute and chronic), acute lymphoblastic leukemia, chronic lymphocytic leukemia, myeloproliferative diseases, multiple myeloma, myelodysplastic syndrome], Hodgkin's disease, non-Hodgkin's lymphoma (malignant lymphoma); Skin: malignant melanoma, basal cell carcinoma, squamous cell carcinoma, Kaposi's sarcoma, nevi, dysplastic nevi, lipoma, angioma, dermatofibroma, keloids, psoriasis; and Adrenal glands: neuroblastoma.

The diagnostic method of the invention provides contacting a biological sample such as a biopsy sample, tissue, cell or fluid (e.g., whole blood, plasma or urine) isolated from a subject with an antibody which binds hypusine-containing eIF-5A. The antibody is allowed to bind to the hypusine-containing eIF-5A antigen to form an antibody-antigen complex. The hypusine-containing eIF-5A antigen, as used herein, includes the mature eIF-5A, a hypusine-containing fragment of mature eIF-5A, or the hypusine amino acid itself. The conditions and time required to form the antibody-antigen complex may vary and are dependent on the biological sample being tested and the method of detection being used. Once non-specific interactions are removed by, for example, washing the sample, the antibody-antigen complex is detected using any one of the immunoassays described above as well a number of well-known immunoassays used to detect and/or quantitate antigens [see, for example, Harlow and Lane, Antibodies: A Laboratory Manual, Cold Spring Harbor Laboratory, New York (1988) 555-612]. Such well-known immunoassays include antibody capture assays, antigen capture assays, and two-antibody sandwich assays. In an antibody capture assay, the antigen is attached to solid support, and labeled antibody is allowed to bind. After washing, the assay is quantitated by measuring the amount of antibody retained on the solid support. In an antigen capture assay, the antibody is attached to a solid support, and labeled antigen is allowed to bind. The unbound proteins are removed by washing, and the assay is quantitated by measuring the amount of antigen that is bound. In a two-antibody sandwich assay, one antibody is bound to a solid support, and the antigen is allowed to bind to this first antibody. The assay is quantitated by measuring the amount of a labeled second antibody that binds to the antigen.

These immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques such as those described by Kennedy, et al. [(1976) Clin. Chim. Acta 70:1-31], and Schurs, et al. [(1977) Clin. Chim Acta 81:1-40].

In accordance with the diagnostic method of the invention, the presence or absence of the antibody-antigen complex is correlated with the presence or absence in the biological sample of the mature eIF-5A antigen, a hypusine-containing peptide fragment thereof, or free hypusine derived from turnover of mature eIF-5A. A biological sample containing elevated levels of said antigen is indicative of a hyperproliferative disorder in a subject from which the biological sample was obtained. Accordingly, the diagnostic method of the invention may be used as part of a routine screen in subjects suspected of having a hyperproliferative disorder or for subjects who may be predisposed to having a hyperproliferative disorder. Moreover, the diagnostic method of the invention may be used alone or in combination with other well-known diagnostic methods to confirm a hyperproliferative disorder.

The diagnostic method of the invention further provides that an antibody of the invention may be used to monitor the levels of hypusine-containing antigen in patient samples at various intervals of drug treatment to identify whether and to which degree the drug treatment is effective in reducing or inhibiting hyperproliferation of cells. Furthermore, hypusine-containing antigen levels may be monitored using an antibody of the invention in studies evaluating efficacy of drug candidates in model systems and in clinical trials. The class of hypusine containing antigens (the free residue, the hypusine containing peptides and mature eIF-5A) provides for surrogate biomarkers in biological fluids to non-invasively assess the global status of cell proliferation. For example, using an antibody of this invention, hypusine-containing antigen levels may be monitored in biological samples of individuals treated with known or unknown therapeutic agents or toxins. This may be accomplished with cell lines in vitro or in model systems and clinical trials, depending on the hyperproliferative disorder being investigated. Persistently increased total levels of hypusine containing antigen in biological samples during or immediately after treatment with a drug candidate indicates that the drug candidate has little or no effect on cell proliferation. Likewise, the reduction in total levels of hypusine antigen indicates that the drug candidate is effective in reducing or inhibiting cell proliferation. This may provide valuable information at all stages of pre-clinical drug development, clinical drug trials as well as subsequent monitoring of patients undergoing drug treatment.

On the other hand, the diagnostic method of the invention also provides a surrogate biomarker to assess rapidly the growth response in clinical situations where administration of a growth promoting drug is therapeutically indicated (eg. to monitor the response of growth hormone administration to children without having to wait to assess the response by an increase in height) or in situations where a growth promoting drug is abused for achieving growth of cell mass without a therapeutic intention (eg. in abuse of growth hormone or erythropoietin for competitive sports) In both situations, rapid measurement of hypusine containing antigen can be done in various biological fluids such as blood, serum or urine.

### Therapeutic Uses of the Antibody Against Mature eIF-5A

Another aspect of the invention provides that an antibody, or a fragment thereof, against a hypusine-containing antigen, may be administered to a human or other animal in an amount to decrease or inhibit cell proliferation. As one may appreciate, any hyperproliferative disorder, which may be diagnosed by an antibody of the invention, may also be treated using an antibody of the invention. A skilled clinician or physician would be able, by routine experimentation, to determine what an effective, non-toxic amount of antibody would be for the purpose of decreasing or inhibiting cell proliferation. Generally, however, an effective dosage will be in the range of about 0.05 to 100 milligrams per kilogram body weight per day.

Furthermore, an antibody of the invention may be administered to a human or other animal in a conventional dosage form prepared by combining an antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

The antibodies of the present invention may also be used to target other chemotherapeutic agents to the site where needed. Alternatively, the antibodies can be used to target radioisotopes to the site where inhibtion of cellular proliferation is desirable. The antibodies may also be employed for diagnostic purposes to identify sites within the patient where the tumor burden is greatest. Furthermore, the antibodies may be used to assess the effectiveness of anti-tumor therapy for prognostic value.

The route of administration of an antibody, or fragment thereof, against the hypusine-containing antigen, may be oral, parenteral, by inhalation or topical. The antibody may be delivered locally to the site of the cancer. The term parenteral as used herein includes intravenous, intramuscular, subcutaneous, rectal, vaginal or intraperitoneal administration. The subcutaneous and intramuscular forms of parenteral administration are generally preferred. The antibody may be delivered using a slow release formulation. It may be delivered in a liposome or a similar device.

The daily parenteral and oral dosage regimens for employing antibodies of the invention to therapeutically decrease cell proliferation will generally be in the range of about 0.05 to 100, but preferably about 0.5 to 10, milligrams per kilogram body weight per day.

An antibody of the invention may also be administered by inhalation. Inhalation, as used herein, includes intranasal and oral inhalation administration. Appropriate dosage forms for such administration, such as an aerosol formulation or a metered dose inhaler, may be prepared by conventional techniques. The preferred dosage amount of an antibody of the invention to be employed is generally within the range of about 10 to 100 milligrams.

An antibody of the invention may also be administered topically. By topical administration is meant non-systemic administration and includes the application of an antibody, or fragments thereof, against hypusine-containing antigens, externally to the epidermis, to the buccal cavity and instillation of such an antibody into the ear, eye and nose, and where it does not significantly enter the blood stream. In a particular topical formulation, the antibody may be effective in treatment of a hyperproliferative condition such as psoriasis. By systemic administration is meant oral, intravenous, intraperitoneal and intramuscular administration. The amount of an antibody required for therapeutic effect will, of course, vary with the antibody chosen, the nature and severity of the condition being treated and the animal undergoing treatment, and is ultimately at the discretion of the physician. A suitable topical dose of an antibody of the invention will generally be within the range of about 1 to 100 milligrams per kilogram body weight daily.

An alternate therapeutic approach for use of the antibodies of the present invention is via insertion of the gene encoding the antibody into a tumor cell whereby the intracellular expression of the antibody gene allows for modulation of the function of the protein for which the antibody is specific. Accordingly, this invention provides for methods and compositions for modulating hypusine-containing eIF-5A function in a cell involving intracellular expression of the antibody described herein that binds to hypusine-containing eIF-5A within the cell, thereby altering the function of this protein. The invention is particularly applicable to inhibiting the expression of hypusinated eIF-5A in a cancer cell, thus inhibiting proliferation and survival of the cell, although the methods of the invention can be similarly used to inhibit the function of other proteins, especially the hypusine forming enzymes, deoxyhypusine synthase and deoxyhypusine hydroxylase (see FIG. 2).

To express an antibody homologue within a cell, a nucleic acid molecule encoding the antibody homologue, such as a recombinant expression vector encoding the antibody homologue, is introduced into the cell. Preferably, the antibody homologue used to modulate protein function is a single chain Fv (scFv) fragment, although whole antibodies, or antigen binding fragments thereof (e.g., Fab fragments) may also be useful.

In a particularly preferred embodiment of the invention, an antibody homologue is expressed intracellularly in a cancerous mammalian cell to inhibit the cell proliferation function of hypusinated eIF-5A. The target cells of interest may be selected from any cell in which hypusinated eIF-5A plays a role in proliferation, such as cancer cells, virally infected B lymphocytes, or antigen activated T cells. A nucleic acid molecule encoding the antibody homologue can be introduced in vivo into cells of interest, by, for example, use of a recombinant viral vector or other vector system suitable for delivery of genes to cells in vivo.

To express an antibody homologue within a cell, a nucleic acid molecule(s) encoding the antibody homologue is prepared and introduced into the cell. An isolated nucleic acid molecule encoding an antibody homologue can be prepared according to standard molecular biology methods using nucleic acid sequences obtained from antibody genes. Isolated nucleic acid molecules encoding antibody chains (or relevant antigen binding portions thereof, such as V_{H} or V_{L} regions), specific for many different particular proteins have been described, and/or are available, in the art. Additionally, such nucleic acids can be isolated by standard techniques, for example, from a hybridoma that expresses a monoclonal antibody specific for a protein of interest, such as mature eIF-5A, or by screening an immunoglobulin expression library (e.g., an immunoglobulin phage display library) with the protein of interest.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the hypusine region of eIF-5A). Such cell lines may be produced, for example, from spleen cells obtained from an immunized animal. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

Alternatively, monoclonal antibodies can be prepared by constructing a recombinant immunoglobulin library, such as a scFv or Fab phage display library and nucleic acid encoding an antibody chain (or portion thereof) can be isolated therefrom. Immunoglobulin light chain and heavy chain first strand cDNAs can be prepared from mRNA derived from lymphocytes of a subject immunized with a protein of interest using primers specific for a constant region of the heavy chain and the constant region of each of the kappa and lambda light chains. Using primers specific for the variable and constant regions, the heavy and light chain cDNAs can then by amplified by PCR. The amplified DNA is then ligated into appropriate vectors for further manipulation in generating a library of display packages. Restriction endonuclease recognition sequences may also be incorporated into the primers to allow for the cloning of the amplified fragment into a vector in a predetermined reading frame for expression on the surface of the display package.

The immunoglobulin library is expressed by a population of display packages, preferably derived from filamentous phage, to form an antibody display library. In addition to commercially available kits for generating phage display libraries (e.g., the Pharmacia Recombinant Phage Antibody System, Catalog No. 27-9400-01; and the Stratagene SurfZAP™. Phage Display Kit, Catalog No. 240612), examples of methods and reagents particularly amenable for use in generating antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) Proc. Natl. Acad. Sci. USA 89:3576-3580; Garrad et al. (1991) Bio/Technology 2:1373-1377; Hoogenboom et al. (1991) Nuc Acid Res 19:41334137; and Barbas et al. (1991) Proc. Natl. Acad. Sci. USA 88:7978-7982. As generally described in McCafferty et al. Nature (1990) 348:552-554, complete VH and VL domains of an antibody, joined by a flexible (Gly₄-Ser)₃ linker, can be used to produce a single chain antibody expressed on the surface of a display package, such as a filamentous phage.

Once displayed on the surface of a display package (e.g., filamentous phage), the antibody library is screened with a protein of interest, ie. mature eIF-5A, or the modifying enzymes DOHS and DOHH, to identify and isolate packages that express an antibody that binds the protein of interest. Display packages expressing antibodies that bind immobilized protein can then be selected. Following screening and identification of a monoclonal antibody (e.g., a monoclonal scFv) specific for the protein of interest, nucleic acid encoding the selected antibody can be recovered from the display package (e.g., from the phage genome) by standard techniques. The nucleic acid so isolated can be further manipulated if desired (e.g., linked to other nucleic acid sequences) and subcloned into other expression vectors by standard recombinant DNA techniques.

Once isolated, nucleic acid molecules encoding antibody chains, or portions thereof, can be further manipulated using standard recombinant DNA techniques. For example, a single chain antibody gene can also be created by linking a VL coding region to a VH coding region via a nucleotide sequence encoding a flexible linker (e.g., (Gly₄-Ser)₃). Single chain antibodies can be engineered in accordance with the teachings of Bird et al. (1988) Science 242:423-426; Huston et al. (1988) Proc. Natl. Acad. Sci USA 85:5879-5883; Ladner, et al. International Publication Number WO 88/06630; and McCafferty, et al. International Publication No. WO 92/10147. A preferred single chain antibody for use in the invention binds to the human hypusine-containing eIF-5A. A plasmid encoding a scFv antibody to hypusine-containing antigen would be prepared using standard molecular biological techniques. Another manipulation that can be performed on isolated antibody genes is to link the antibody gene to a nucleotide sequence encoding an amino acid sequence that directs the antibody homologue to a particular intracellular compartment. A preferred nucleotide sequence to which an antibody gene is linked encodes a signal sequence (also referred to as a leader peptide). Signal sequences are art-recognized amino acid sequences that direct a protein containing the signal sequence at its amino-terminal end to the endoplasmic reticulum (ER). Typically, signal sequences comprise a number hydrophobic amino acid residues. Alternatively, an antibody homologue can be linked to an amino acid sequence that directs the antibody homologue to a different compartment of the cell. For example, a nuclear localization sequence (NLS) can be linked to the antibody homologue to direct the antibody homologue to the cell nucleus. Nuclear localization sequences are art-recognized targeting sequences. Typically, an NLS is composed of a number of basic amino acid residues.

Following isolation of antibody genes, as described above, and, if desired, further manipulation of the sequences, DNA encoding the antibody homologue can be inserted into an expression vector to facilitate transcription and translation of the antibody coding sequences in a host cell. Within the expression vector, the sequences encoding the antibody homologue are operatively linked to transcriptional and translational control sequences. These control sequences include promoters, enhancers and other expression control elements (e.g., polyadenylation signals). Such regulatory sequences are known to those skilled in the art and are described in Goeddel, Gene Expression Technology: Methods in Enzymology 185, Academic Press, San Diego, Calif. (1990). The expression vector and expression control sequences are chosen to be compatible with the host cell used. Expression vectors can be used to express one antibody chain (e.g., a single chain antibody) or two antibody chains (e.g., a Fab fragment). To express two antibody chains, typically the genes for both chains are inserted into the same expression vector but linked to separate control elements.

Expression of a nucleic acid in mammalian cells is accomplished using a mammalian expression vector. When used in mammalian cells, the expression vector's control functions are often provided by viral material. For example, commonly used promoters are derived from polyoma, Adenovirus 2, cytomegalovirus (CMV) and Simian Virus 40. An example of a suitable mammalian expression vector is pCDNA3 (commercially available from Invitrogen), which drives transcription via the CMV early intermediate promoter/enhancer and contains a neomycin resistance gene as a selective marker. Other examples of mammalian expression vectors include pCDM8 (Seed, B., (1987) Nature 329:840) and pMT2PC (Kaufman et al. (1987), EMBO J 6:187-195). Alternative to the use of constitutively active viral regulatory sequences, expression of an antibody homologue gene can be controlled by a tissue-specific regulatory element that directs expression of the nucleic acid preferentially in a particular cell type. Tissue-specific regulatory elements are known in the art.

In one embodiment, a recombinant expression vector of the invention is a plasmid vector. Plasmid DNA can be introduced into cells by a variety of techniques either as naked DNA or, more commonly, as DNA complexed with or combined with another substance. Alternatively, in another embodiment, the recombinant expression vector of the invention is a virus, or portion thereof, which allows for expression of a nucleic acid introduced into the viral nucleic acid. For example, replication defective retroviruses, adenoviruses and adeno-associated viruses can be used for recombinant expression of antibody homologue genes. Virally-mediated gene transfer into cells can be accomplished by infecting the target cell with the viral vector.

Non-limiting examples of techniques which can be used to introduce an expression vector encoding an antibody homologue into a host cell include:

Adenovirus-Polylysine DNA Complexes: Naked DNA can be introduced into cells by complexing the DNA to a cation, such as polylysine, which is then coupled to the exterior of an adenovirus virion (e.g., through an antibody bridge, wherein the antibody is specific for the adenovirus molecule and the polylysine is covalently coupled to the antibody) (see Curiel, D. T., et al. (1992) Human Gene Therapy 3:147-154). Entry of the DNA into cells exploits the viral entry function, including natural disruption of endosomes to allow release of the DNA intracellularly. A particularly advantageous feature of this approach is the flexibility in the size and design of heterologous DNA that can be transferred to cells.

Receptor-Mediated DNA Uptake: Naked DNA can also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J Biol. Chem. 267:963-967; and U.S. Pat. No. 5,166,320). Binding of the DNA-ligand complex to the receptor facilitates uptake of the DNA by receptor-mediated endocytosis. Receptors to which a DNA-ligand complex have targeted include the transferrin receptor and the asialoglycoprotein receptor. Additionally, a DNA-ligand complex can be linked to adenovirus capsids which naturally disrupt endosomes, thereby promoting release of the DNA material into the cytoplasm and avoiding degradation of the complex by intracellular lysosomes (see for example Curiel et al. (1991) Proc. Natl. Acad. Sci. USA 88:8850; and Cotten, M. et al. (1992) Proc. Natl. Acad. Sci. USA 89:6094-6098; Wagner, E. et al. (1992) Proc. Natl. Acad. Sci. USA 89:6099-6103). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Liposome-Mediated transfection (“lipofection”): Naked DNA can be introduced into cells by mixing the DNA with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel, F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J Med. Sci. 298:278; and Gould-Fogerite et al. (1989) Gene 84:429-438.

Direct Injection: Naked DNA can be introduced into cells by directly injecting the DNA into the cells. For an in vitro culture of cells, DNA can be introduced by microinjection, although this not practical for large numbers of cells. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BioRad).

Retroviral Mediated Gene Transfer: Defective retroviruses are well characterized for use in gene transfer for gene therapy purposes (for a review see Miller, A. D. (1990) Blood 76:271). A recombinant retrovirus can be constructed having a nucleic acid encoding a gene of interest (e.g., an antibody homologue) inserted into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art.

Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Adenoviral Mediated Gene Transfer: The genome of an adenovirus can be manipulated such that it encodes and expresses a gene product of interest (e.g., an antibody homologue) but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to many other gene delivery vectors (Berkner et al. cited supra; Haj-Ahmand and Graham (1986) J Virol. 57:267). Most replication-defective adenoviral vectors currently in use are deleted for all or parts of the viral E1 and E3 genes but retain as much as 80% of the adenoviral genetic material.

Adeno-Associated Viral Mediated Gene Transfer: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J Respir. Cell. Mol. Biol. 7:349-356; Samulski et al. (1989) J Virol. 63:3822-3828; and McLaughlin et al. (1989) J Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J Virol. 51:611-619; and Flotte et al. (1993) J Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of the introduced DNA can be detected, for example, by Northern blotting, RNase protection or reverse transcriptase-polymerase chain reaction (RT-PCR). Expression of the introduced gene product (e.g., the antibody homologue) in the cell can be detected by an appropriate assay for detecting proteins, for example by immunohistochemistry.

As will be appreciated by those skilled in the art, the choice of expression vector system will depend, at least in part, on the host cell targeted for introduction of the nucleic acid. For example, nucleic acid encoding an antibody homologue to hypusine-containing antigen (e.g., anti-eIF-5A scFv) is preferably introduced into tumor cells overexpressing hypusine-containing eIF-5A. Tumor cells known to overexpress hypusine-containing eIF-5A include epithelial carcinoma cells, genitourinary cancers, carcinoma cells derived from tissues or organs including breast, ovary, lung, and gastrointestinal tract. Preferred expression vectors and delivery systems for introducing nucleic acid into malignant cells include transfection with adenoviral-polylysine DNA complexes and adenoviral vector-mediated gene transfer. These delivery systems are suitable for introduction of nucleic acid into cells in vitro, or more preferably for tumor cells, in vivo.

The functional outcome of intracellular antibody expression, e.g. scFv against mature eIF-5A, on the subsequent expression and/or function of the protein targeted for antibody binding (referred to as the target protein, in this application, mature eIF-5A but obviously also the two hypusine-forming enzymes, deoxyhypusine synthase and deoxyhypusine hydroxylase) can be assessed by suitable assays that monitor the expression and/or function of the target protein, including standard immunohistochemistry or immunoelectron microscopy techniques.

Alternatively, cell proliferation can be measured using commercially available cell proliferation assays. The functional outcome of intracellular antibody homologue expression targeting mature eIF-5A on tumor cell growth and survival, or on the expansion of immunocompetent cells with unwanted specificity in a mammal can be assessed in vivo using animal model systems that may be predictive of therapeutic efficacy in humans. For example, the antibody genes may be inserted into a human cancer cell known to contain hypusinated eIF-5A. These cells may be implanted into athymic nude mice, and tumor growth may be monitored visually over time.

### Pharmaceutical Compositions

While it is possible for an antibody or fragment thereof to be administered alone, it is preferable to present it as a pharmaceutical composition. A generally recognized compendium of methods and ingredients of pharmaceutical compositions is Remington's Pharmaceutical Science, 15th ed., Mack Publishing Company, Baston, Pa. The active ingredient may comprise, for topical administration, from 0.001% to 10% w/w, e.g., from 1% to 2% by weight of the composition, although it may comprise as much as 10% w/w but preferably not in excess of 5% w/w and more preferably from 0.1% to 1% w/w of the composition.

Topical compositions of the invention, may comprise an antibody of the invention together with one or more acceptable carrier(s) and optionally any other therapeutic ingredients(s). The carrier(s) must be acceptable in the sense of being compatible with the other ingredients of the composition and not deleterious to the recipient thereof.

Compositions suitable for topical administration include liquid or semi-liquid preparations suitable for penetration through the skin to the site of where treatment is required, such as liniments, lotions, creams, ointments or pastes, and drops suitable for administration to the eye, ear or nose.

Drops may comprise sterile aqueous or oily solutions or suspensions and may be prepared by dissolving an antibody of the invention in a suitable aqueous solution of a bactericidal and/or fungicidal agent and/or any other suitable preservative, and preferably including a surface active agent. The resulting solution may then be clarified by filtration, sterilized by filtration and transferred to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the invention are semi-solid compositions of an antibody, or a fragment thereof, against mature eIF-5A for external application. They may be made by mixing an antibody in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The composition may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

The antibodies and pharmaceutical compositions of the invention are particularly useful for parenteral administration, i.e., subcutaneously, intramuscularly or intravenously. The compositions for parenteral administration will commonly comprise a solution of an antibody or fragment thereof of the invention or a cocktail thereof dissolved in an acceptable carrier, preferably an aqueous carrier. A variety of aqueous carriers may be employed, e.g., water, buffered water, 0.4% saline, 0.3% glycine, and the like. These solutions are sterile and generally free of particulate matter. These solutions may be sterilized by conventional, well-known sterilization techniques. The compositions may contain pharmaceutically acceptable auxiliary substances as required to approximate physiological conditions such as pH adjusting and buffering agents, etc. The concentration of the antibody or fragment thereof of the invention in such pharmaceutical compositions may vary widely, i.e., from less than about 0.5%, usually at or at least about 1% to as much as 15 or 20% by weight, and will be selected primarily based on fluid volumes, viscosities, etc., according to the particular mode of administration selected.

Thus, a pharmaceutical composition of the invention for intramuscular injection could be prepared to contain 1 ml sterile buffered water, and 50 mg of an antibody or fragment thereof of the invention. Similarly, a pharmaceutical composition of the invention for intravenous infusion could be made up to contain 250 ml of sterile Ringer's solution, and 150 mg of an antibody or fragment thereof of the invention. Actual methods for preparing parenterally administrable compositions are well-known or will be apparent to those skilled in the art, and are described in more detail in, e.g., Remington's Pharmaceutical Science, 15th ed., Mack Publishing Company, Easton, Pa.

The antibodies, or fragments thereof, of the invention may be lyophilized for storage and reconstituted in a suitable carrier prior to use. This technique is effective with conventional immunoglobulins and art-known lyophilization and reconstitution techniques may be employed.

The pharmaceutical composition of the invention may be administered for prophylactic and/or therapeutic treatments. In therapeutic application, compositions are administered to a subject already suffering from a hyperproliferative disorder, in an amount sufficient to cure or at least partially arrest the disorder and its complications. In prophylactic applications, compositions containing the present antibodies or fragments thereof are administered to a subject not already in a disease state but one that may be predisposed to a hyperprolifertive disorder to enhance the subject's resistance.

It will be recognized by one of skill in the art that the optimal quantity and spacing of individual dosages of an antibody or fragment thereof of the invention will be determined by the nature and extent of the hyperproliferative disorder being treated, the form, route and site of administration, and the particular animal being treated, and that such optimums may be determined by conventional techniques. It will also be appreciated by one of skill in the art that the optimal course of treatment, i.e., the number of doses of an antibody or fragment thereof of the invention given per day for a defined number of days, may be ascertained by those skilled in the art using conventional course of treatment determination tests.

### Screening Assays

A still further aspect of the invention relates to screening assays to identify agents which inhibit or displace the binding of an antibody against mature eIF-5A to the hypusine-containing region of eIF-5A. The invention encompasses both in vivo and in vitro assays to screen small molecules, compounds, recombinant proteins, peptides, nucleic acids, antibodies, etc. which bind mature eIF-5A and thus, identify potential therapeutic agents for the treatment of hyperproliferative disorders.

In a preferred embodiment, the binding of the agent is determined through the use of competitive binding assays. The competitor is an antibody of the invention known to bind to the hypusine-containing eIF-5A protein. Competitive screening assays may be done by combining the hypusine-containing eIF-5A protein and an antibody of the invention in a first sample. A second sample comprises an test agent, hypusine-containing eIF-5A and an antibody of the invention. The binding of the antibody is determined for both samples, and a change, or difference in binding between the two samples indicates the presence of a test agent capable of binding to hypusine-containing eIF-5A and potentially modulating its activity. That is, if the binding of the antibody is different in the second sample relative to the first sample, the test agent is capable of binding to hypusine-containing eIF-5A protein. Similar designs that utilize antibodies of this invention for the identification of non-antibody compounds that bind to mature eIF-5A are obvious to those skilled in the art.

One variation provides that the agent is labeled. Either the agent, or the competitor, or both, is added first to hypusine-containing eIF-5A protein for a time sufficient to allow binding. Incubations may be performed at any temperature which facilitates optimal activity, typically between 4 and 40° C. Incubation periods are selected for optimum activity, but may also be optimized to facilitate rapid high-throughput screening. Typically between 0.1 and 1 hour will be sufficient. Excess reagent is generally removed or washed away. The second component is then added, and the presence or absence of the labeled component is followed, to indicate binding.

It is preferred that the competitor is added first, followed by the test agent. Displacement of the competing antibody of this invention is an indication that the test agent is binding to mature eIF-5A protein and thus is capable of binding to, and potentially modulating, the activity of hypusine-containing eIF-5A protein. In this reaction either component may be labeled. Thus, for example, if the competitor is labeled, the presence of label in the wash solution indicates displacement by the agent. Alternatively, if the test agent is labeled, the presence of the label on the support indicates displacement.

Alternatively, the test agent is added first, with incubation and washing, followed by the competitor. The absence of binding by the competitor may indicate the test agent is bound to mature eIF-5A protein with a higher affinity. Thus, if the agent is labeled, the presence of the label on the support, coupled with a lack of competitor binding, may indicate the test agent is capable of binding to hypusine-containing eIF-5A protein.

Agents encompass numerous chemical classes, though typically they are organic molecules, preferably small organic compounds having a molecular weight of more than 100 and less than about 3,500 daltons. Agents comprise functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups. Agents may also be found among biomolecules including peptides, saccharides, fatty acids, steroids, purines, pyrimidines, derivatives, structural analogs or combinations thereof.

Agents are obtained from a wide variety of sources including libraries of synthetic or natural compounds.

Alternatively, the antibodies of this invention may be used for the design and synthesis of either peptide or non-peptide compounds (mimetics) that specifically bind to hypusine-containing eIF-5A [see, e.g., Saragovi, et al (1991) Science 253:792-795].

The assays provided use mature eIF-5A protein. Alternatively, fragments of the hypusine-containing eIF-5A protein may be used. For example, the region of mature eIF-5A which is homologous to dihydrofolate reductase and/or contains the hypusine residue may be used, or the region homologous to cold-shock protein A may be used. In addition, the assays described herein may use either isolated mature eIF-5A or cells or animal models relying on hypusine-containing eIF-5A.

A variety of other reagents may be included in the screening assays. These include reagents like salts, neutral proteins, e.g. albumin, detergents, etc. which may be used to facilitate optimal protein-protein binding and/or reduce non-specific or background interactions. Also, reagents that otherwise improve the efficiency of the assay, such as protease inhibitors, nuclease inhibitors, anti-microbial agents, and the like may be used. The mixture of components may be added in any order that provides for the requisite binding.

The methods of the invention are used to identify compounds, which inhibit or displace an antibody that binds to mature eIF-5A and are therefore useful in the treatment of hyperproliferative disorders. Hyperproliferative disorders which can be treated by the methods and compositions provided herein include, but are not limited to, cancers (as described above), autoimmune disease, restenosis, arthritis, graft rejection, inflammatory bowel disease, proliferation induced after medical procedures, including, but not limited to, surgery, angioplasty, and the like.

In the same manner that an antibody of the invention may be administered to treat a hyperproliferative disorder, so may an agent that inhibits or displaces an antibody of this invention be administered to treat a hyperproliferative disorder. Accordingly, a further aspect of the invention provides methods for decreasing cell proliferation by administering to a subject with a hyperproliferative disorder an agent which binds to and therefore blocks the functionally significant hypusine region of mature eIF-5A. As one of skill in the art may appreciate, the pharmaceutical compositions comprising the agent and a pharmaceutically acceptable carrier, as well as the route of administration of such pharmaceutical compositions, would be similar to those provided above for an antibody of this invention. The optimal quantity and spacing of individual dosages of an agent of the invention will be determined by the nature of the agent, the nature and extent of the hyperproliferative disorder being treated, the form, route and site of administration, and the particular animal being treated. Such optimums may be determined by conventional techniques of monitoring cell proliferation.

### EXAMPLES

The following examples are put forth so as to provide those of ordinary skill in the art with a complete disclosure and description of how to make and use the ligands described herein for diagnostic and therapeutic applications, and to provide a suitable means for identifying novel anti-folates that modulate control of gene expression executed by eIF-5A and development of pharmaceutical compositions for therapeutic use, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Furthermore, for those knowledgeable in the art, the outlined results represent information directly enabling i) the detection of proliferating cells in histology sections; ii) the therapeutic control of mature eIF-5A as required for cell proliferation and retroviral replication; and iii) the high-throughput screening for natural and/or man-made compounds that target the hypusine region of mature eIF-5A; and iv) the rationale for development of novel anti-folates for the indications provided herein based on the supportive data.

### Example 1

### Production of NIH-353 Polyclonal Antibody

Human mature eIF-5A protein, known to contain the hypusine region of mature eIF-5A, was isolated as described (Park M H, et al. (1986), J. Biol. Chem. 261,14515 -14519). Polyclonal antiserum against purified human hypusine-containing eIF-5A was generated in rabbits using standard techniques known to those skilled in the art.

### Example 2

### Characterization of NIH-353 as Specifically Directed Against the Hypusine Region Of Mature eIF-5A

To establish the antigen specificity of NIH-353, decreasing concentrations of the three biosynthetic forms of eIF-5A [protein as encoded by the eIF-5A genes [eIF-5A (Lys)]); protein representing the half-product formed during post-translational modification [eIF-5A (Dhp)]; and protein representing the final product formed by post-translational modification, i.e. the hypusine-containing (mature) eIF-5A [eIF-5A (Hpu)] were studied side-by-side on Western blots, using a commercially available product (NuPage™ Bis-Tris Electrophoresis System; Invitrogen Life Technologies, Carlsbad, Calif.). NIH 353, the primary antibody, was diluted 1/1000 in TTBS (0.15 M NaCl, 0.01 M Tris, 0.0.05% Tween) with 0.5% milk proteins. After multiple washes in TTBS, the membrane was agitated in TTBS/0.5% milk proteins containing the secondary anti-rabbit antibody, diluted 1/40000. Signal was developed with a commercial chemoluminescence reagent (Renaissance™; NEN, Boston, Mass.) on commercial film (BioMax MR™; Kodak, Rochester, N.Y.). Results are summarized in FIG. 3.

### Example 3

### Characterization of NIH-353 as Selectively Reacting with Proliferating Cells in Human Tissues

To establish the staining selectivity of NIH-353, we used immunocytochemical methods as published (Dabbs, DJ Diagnostic Immunohistochemistry. Churchill Livingston, Philadelphia, 2002). Employing physical parameters that we have optimized for work with NIH-353, antigen retrieval was performed in a commercially available liquid (Citra™; BioGenex, San Ramon, Calif.), and involves cycled microwave irradiation at temperatures above 94.7° C. We used the streptavidin-biotin/horseradish peroxidase complex technique, with diaminobenzidine as chromogen and hematoxylin as counterstain. Formalin-fixed paraffin-embedded human tissues were sectioned to contain at least one proliferative, anatomically defined area. FIG. 4 summarizes representative findings for a set of normal human tissues, FIG. 8 summarizes representative findings of human tissues containing neoplastic pre-invasive cells, still confined to the epithelial layer. Similar staining pattern as shown in FIG. 8, Panel A2, was found with several invasive epithelial neoplasias. Example given of the vulva, the cervix, the uterus and the ovaries.

### Example 4

### The Crystal Structure

Recent structural analyses of eIF-5A indicate that its C-terminal part folds like the cold-shock protein A of E. coli, which prevents mRNA duplex formation at low temperatures (Peat, T. S. et al, Structure 6, 1207-1214, 1998), and that human eIF-5As in their most N-terminal part contain motifs II, III, IV, and V of ATP-utilizing mRNA helicases, required for unwinding of mRNA duplexes (Hannauske-Abel, H. et al., FASEB J 16, A549; 2002). Hypothesizing that mature eIF-5A crystal structure/sequence data contain further clues for its interaction with specific mRNAs essential for cell cycle control, we refined parameters and strategies of an exhaustive database analysis. Using the spatial coordinates of only the N-terminal part of eIF-5A of M. jannaschii (PDB# 1EIP), we noted a significant homology with the crystal structure of plasmid-encoded dihydrofolate reductase (DHFR) of E. coli (PDB# 1vie), using the Dali algorithm (Z score=4.4), see FIG. 5 (Hannauske-Abel, H. et al., Eur. J. Cancer 38, Supplement 7: S105, 2002).

### Example 5

### Sequence Based Evidence for the Folate Region of eIF-5A

Optimized sequence alignment between human DHFR (Acc.# XM_{—}165390) and the human eIF-5As (1: Acc.# NP_{—}001961; 2: Acc.# NP_{—}065123) revealed 37% identify/similarity with eIF-5A-1 and 35% identify/similarity with eIF-5A-2. The N-terminal REGION of the human eIF-5As displays several isolocated residues that in dihydrofolate reductase (DHFR) participate in binding of folate and methotrexate (e.g. Ile⁷, Pro⁶¹, Arg⁷⁰) and of NADPH (e.g. Gly²⁰, Lys⁵⁴, Gly¹¹⁷, Ser¹¹⁸), whereas distinct differences, such as the E30Q isolocation, suggest limited DHFR activity, see FIG. 6 (Hannauske-Abel, H. et al., Eur. J. Cancer 38, Supplement 7: S105, 2002).

### Example 6

### Effect of a Hypusine Inhibitor on Cellular DOHH Activity and on Cell Cycle Progression

The eukaryotic translation initiation factor 5A (hypusine-containing eIF-5A) exists in two genetically distinct variants, 1 and 2. Both contain a single hypusine residue, formed by posttranslational modification within a -Gly-X-Y-Gly- collagen motif known to fold into a B-turn. Like the collagens, the eIF-5As are subject to posttranslational protein hydroxylation by a 2-oxoacid-utilizing dioxygenase; the eIF-5A-hydroxylating enzyme is the hypusine-forming deoxyhypusyl hydroxylase (DOHH). The catalytic cycle of all 2-oxoacid-utilizing dioxygenases including DOHH, follows a unique pathway formulated by Hanauske-Abel et al. (Hanauske-Abel, H. M., (1995) FEBS Lett 366, 92-98), which proceeds at their non-heme ferrous ion as a ligand reaction between a chelating 2-oxoacid moiety and an end-on coordinated dioxygen unit to generate the reactive iron-oxo species essential for product formation. The detailed orbital interactions specified by the HAG mechanism were essential for the rational discovery of inhibitors that suppress hypusine formation in eIF-5As, revealing an essential role in polysomal loading of specific mRNAs (hymns ) and in the onset of DNA replication, i.e. exit from G1 (Hanauske-Abel, H. M., et al, (1994), Biochim Biophys. Acta 1221, 115-124). FIG. 7 shows the typical effect of a hypusine inhibitor on cellular DOHH activity and on cell cycle progression. Thus, the hypusine-containing eIF-5As and their unique hypusine residue appear closely related to, or even represent the molecular equivalent of, one of the major restriction points in the cell cycle: the irreversible commitment to initiate DNA replication. The hypusine region of mature eIF-5A has emerged as the target for several experimental cytostatic agents and the antifungal compound ciclopirox (Clement, P. M. J. et al (2002), International I. Cancer 100: 491498).

### Example 7

### Antibody Characterization

Applicants recently characterized an antibody, NIH-353, as being reactive with human hypusine-containing eIF-5A (FIG. 3). The hypusine residue is not genetically encoded; rather, it derives from a genetically encoded lysine moiety, after butylamine transfer utilizing spermidine, followed by the DOHH-mediated hydroxylation utilizing atmospheric oxygen (FIG. 2). Immunohistochemical analysis of human tissues and cancers has confirmed that the hypusine region-selective NIH-353 labels only proliferating cells, as shown in FIG. 4 for normal endometrium (proliferative phase) [compare to stain with Ki-67 antibody, routinely used for detection of proliferating cells in human tissue FIG. 4]. Note that Ki-67 stain is located to the nuclei of cells, produces a punctate stain, whereas NIH353 is exclusively located to the cytoplasm of cells and therefore gives a non-punctate, homogeneous staining pattern.

### Materials and Methods

### Protein Structural Alignments

Protein structural alignments used the crystal coordinates of eIF-5A from M. jannaschii (PDB# 2EIF), the plasmid-encoded dihydrofolate reductase of E. coli (PDB# 1VIE and PDB#1VIF), and the cold-shock protein of E. coli (PDB# 1MJC). Comparisons were performed with Dali v. 2. Molecules were visualized with InsightII. Linear alignments were generated with ClustalW and manually optimized.

### Western Blots

Western blots were performed after gel electrophoresis and transfer to nitrocellulose membranes, using a commercially available product (NuPage™ Bis-Tris Electrophoresis System; Invitrogen Life Technologies, Carlsbad, Calif.). NIH 353, the primary antibody, was diluted 1/1000 in TTBS (0.15 M NaCl, 0.01 M Tris, 0.0.05% Tween) with 0.5% milk proteins. After multiple washes in TTBS, the membrane was agitated in TTBS/0.5% milk proteins containing the secondary anti-rabbit antibody, diluted 1/40000. Signal was developed with a commercial chemoluminescence reagent (Renaissance™; NEN, Boston, Mass.) on commercial film (BioMax MR™; Kodak, Rochester, N.Y.).

### Immunohistochemical Analysis of Tissues

To establish the staining selectivity of NIH-353, we used immunocytochemical methods as published (Dabbs, DJ Diagnostic Immunohistochemistry. Churchill Livingston, Philadelphia, 2002), employing physical parameters that we have optimized for work with NIH-353. Antigen retrieval was performed in a commercially available liquid (Citra™; BioGenex, San Ramon, Calif.), and involves cycled microwave irradiation at temperatures above 94.7° C. We used the streptavidin-biotin/horseradish peroxidase complex technique, with diaminobenzidine as chromogen and hematoxylin as counterstain. Formalin-fixed paraffin-embedded human tissues were sectioned to contain at least one proliferative, anatomically defined area.

What is claimed is:

1. A ligand binding to the hypusine region of eukaryotic intiation factor 5A, said hypusine region comprising residues 35 to 65 of the human eIF-5A amino acid sequence as in SEQ ID NOs: 1 and 2, wherein the binding of said ligand in biological samples results in a detectable signal for identification of hypusine-containing eIF-5A and its hypusine-containing fragments.

2. The ligand of claim 1, wherein said ligand comprises an antibody, or an eIF-5A-binding derivative or fragment thereof, and wherein said antibody recognizes a hypusine containing eIF-5A molecule, and binds to a hypusine-deficient eIF-5A molecule in an amount of up to about 5% of the extent of binding to the hypusine-containing eIF-5A molecule.

3. The ligand of claim 2, wherein said ligand specifically binds to a human hypusine-containing eIF-5A molecule, and wherein said binding occurs if said human eIF-5A contains hypusine.

4. A method for distinguishing proliferating cells from non-proliferating cells in a specimen of biological fluid or tissue, said method comprising:
a. Processing a specimen of biological fluid or tissue to yield a mixture of cells, said mixture consisting of proliferating and non-proliferating cells present in the biological fluid or tissue; and
b. Treating said mixture of cells with a fixing agent to permeabilize and fix said cells; and
c. Reacting the cells with a ligand of claim 1, wherein said ligand specifically binds to the hypusine-containing region of eIF-5A; and
d. Separating said cells from unreacted ligand of step c; and
e. Detecting said ligand remaining within the fixed cells, whereby detection of said ligand is accomplished by use of a reagent selected from the group consisting of a radiolabel, an enzyme, a chromophore and a fluorescer,
wherein the detecting of said ligand indicates the presence of proliferating cells.

5. The method of claim 4, further comprising depositing said specimen on a solid support and detecting the ligand within the cells of said specimen, wherein said detecting is accomplished using a microscope.

6. The method of claim 4, further comprising maintaining said specimen in suspension and detecting the ligand within the cells of said specimen, wherein said detecting is accomplished using a flow cytometer.

7. A method for distinguishing proliferating cells from non-proliferating cells in a specimen of biological fluid or tissue, said method comprising:
a. Processing a specimen of biological fluid or tissue to yield a mixture of cells, said mixture consisting of proliferating and non-proliferating cells present in the biological fluid or tissue; and
b. Treating said mixture of cells with a fixing agent to permeabilize and fix said cells; and
c. Reacting said cells with a ligand of claim 2, wherein said ligand recognizes the hypusine containing region of eIF-5A; and
d. Separating said cells from unreacted ligand of step c; and
e. Detecting said ligand remaining within the fixed cells, whereby detection of said ligand is accomplished by use of a reagent selected from the group consisting of a radiolabel, an enzyme, a chromophore and a fluorescer,
wherein the detecting of said ligand indicates the presence of proliferating cells.

8. The method of claim 7, further comprising depositing said specimen on a solid support and detecting the ligand within the cells of said specimen, wherein said detecting is accomplished using a microscope.

9. The method of claim 7, further comprising maintaining said specimen in suspension and detecting the ligand within the cells of said specimen, wherein said detecting is accomplished using a flow cytometer.

10. A method for distinguishing proliferating cells from non-proliferating cells in a specimen of biological fluid or tissue, said method comprising:
a) Processing a specimen of biological fluid or tissue to yield a mixture of cells, said mixture consisting of proliferating and non-proliferating cells present in the biological fluid or tissue; and
b) Treating said mixture of cells with a fixing agent to permeabilize and fix said cells; and
c) Reacting said cells with a ligand of claim 3, wherein said ligand recognizes the hypusine containing region of eIF-5A; and
d) Separating said cells from unreacted ligand of step c; and
e) Detecting said ligand remaining within the fixed cells, whereby detection of said ligand is accomplished by use of a reagent selected from the group consisting of a radiolabel, an enzyme, a chromophore and a fluorescer,
wherein the detecting of said ligand indicates the presence of proliferating cells.

11. The method of claim 10, further comprising depositing said specimen on a solid support and detecting the ligand within the cells of said specimen, wherein said detecting is accomplished using a microscope.

12. The method of claim 10, further comprising maintaining said specimen in suspension and detecting the ligand within the cells of said specimen, wherein said detecting is accomplished using a flow cytometer.

13. A method of diagnosing a hyperproliferative disorder comprising contacting a biological sample with a ligand of claim 1 and detecting said ligand bound to eIF-5A in the sample, wherein the detection of ligand bound to hypusine containing eIF-5A is indicative of a hyperproliferative disorder.

14. A method of diagnosing a hyperproliferative disorder comprising contacting a biological sample with a ligand of claim 2 and detecting said ligand bound to hypusine-containing eIF-5A in the sample, wherein the detection of ligand bound to hypusine-containing eIF-5A is indicative of a hyperproliferative disorder.

15. A method of diagnosing a hyperproliferative disorder comprising contacting a biological sample with a ligand of claim 3 and detecting said ligand bound to hypusine-containing eIF-5A in the sample, wherein the detection of ligand bound to hypusine-containing eIF-5A is indicative of a hyperproliferative disorder.

16. A method of diagnosing intraepithelial neoplasia comprising contacting a biological sample with a ligand of claim 1 and detecting said ligand bound to hypusine-containing eIF-5A in the sample, wherein the detection of ligand bound to hypusine-containing eIF-5A is indicative of local neoplasia.

17. A method of diagnosing intraepithelial neoplasia comprising contacting a biological sample with a ligand of claim 2 and detecting said ligand bound to hypusine-containing eIF-5A in the sample, wherein the detection of ligand bound to hypusine-containing eIF-5A is indicative of local neoplasia.

18. A method of diagnosing intraepithelial neoplasia comprising contacting a biological sample with a ligand of claim 3 and detecting said ligand bound to hypusine-containing eIF-5A in the sample, wherein the detection of ligand bound to hypusine-containing eIF-5A is indicative of local neoplasia.

19. A method of diagnosing intraepithelial neoplasia comprising contacting a biopsy containing epithelium with a ligand of claim 1 and detecting any of said ligand bound to hypusine-containing eIF-5A in the sample, wherein the detection of ligand bound to hypusine-containing eIF-5A is indicative of local neoplasia.

20. A method of diagnosing intraepithelial neoplasia comprising contacting a biopsy containing epithelium with a ligand of claim 2 and detecting any of said ligand bound to hypusine-containing eIF-5A in the sample, wherein the detection of ligand bound to hypusine-containing eIF-5A is indicative of local neoplasia.

21. A method of diagnosing intraepithelial neoplasia comprising contacting a biopsy containing epithelium with a ligand of claim 3 and detecting any of said ligand bound to hypusine-containing eIF-5A in the sample, wherein the detection of ligand bound to hypusine-containing eIF-5A is indicative of local neoplasia.

22. A method for determining in a biological sample the concentration of hypusine containing eIF-5A and/or its hypusine-containing fragments, wherein the hypusine region of said protein is located on residues 35 to 65 of the human eIF-5A as in SEQ ID NOs: 1 and 2, comprising:
a) contacting said sample with a ligand of claim 1, under conditions wherein said ligand can form a complex with hypusine contained in the sample either as a free amino acid or bound within the hypusine region of eIF-5A or its fragments; and
b) determining the amount of hypusine-containing antigen bound by said ligand by detecting the amount of complex formed, wherein said detecting is accomplished by use of a reagent selected from the group consisting of a radiolabel, an enzyme, a chromophore and a flourescer.

23. A method for determining in a biological sample the concentration of hypusine containing eIF-5A and/or its hypusine-containing fragments, wherein the hypusine region of said protein is located on residues 35 to 65 of the human eIF-5A as in SEQ ID NOs: 1 and 2, comprising:
a) contacting said sample with a ligand of claim 2, under conditions wherein said ligand can form a complex with hypusine contained in the sample either as a free amino acid or bound within the hypusine region of mature eIF-5A or its fragments; and
b) determining the amount of hypusine-containing antigen bound by said ligand by detecting the amount of complex formed, wherein said detecting is accomplished by use of a reagent selected from the group consisting of a radiolabel, an enzyme, a chromophore and a flourescer.

24. A method for determining in a biological sample the concentration of hypusine containing eIF-5A and/or its hypusine-containing fragments, wherein the hypusine region of said protein is located on residues 35 to 65 of the human eIF-5A as in SEQ ID NOs: 1 and 2, comprising:
a. contacting said sample with a ligand of claim 3, under conditions wherein said ligand can form a complex with hypusine contained in the sample either as a free amino acid or bound within the hypusine region of mature eIF-5A or its fragments; and
b. determining the amount of hypusine-containing antigen bound by said ligand by detecting the amount of complex formed, wherein said detecting is accomplished by use of a reagent selected from the group consisting of a radiolabel, an enzyme, a chromophore and a flourescer.

25. A method for inhibiting in a cell the biological activity of the hypusine region of mature eIF-5A that corresponds to amino acid residues 35 to 65 of human eIF-5A as in SEQ ID NOs: 1 and 2, comprising:
a) introducing into said cell of a patient in need of such treatment a nucleic acid molecule encoding an antibody homologue, or a derivative or fragment thereof; wherein said antibody homologue, derivative or fragment thereof is specifically reactive to the hypusine region of mature eIF-5A; and
b) wherein said antibody homologue is expressed intracellularly and binds to said hypusine region intracellularly thereby inhibiting the biological activity of the hypusine region of mature eIF-5A.

26. The method of claim 25, wherein the antibody homologue is a single chain Fv fragment.

27. The method of claim 25, wherein the nucleic acid molecule is a recombinant expression vector selected from the group consisting of viral vectors and plasmid vectors.

28. A method of identifying a therapeutic agent that decreases the biological activity of the hypusine region of mature eIF-5A, comprising contacting hypusine-containing eIF-5A with an agent and detecting the binding of an antibody of claim 2 to hypusine-containing eIF-5A, wherein said method is conducted by high throughput screening.

29. A method according to claim 25, wherein the high throughput screening of the biological activity of the hypusine region of mature eIF-5A is directed at cell proliferation.

30. A method according to claim 25, wherein the high throughput screening of the biological activity of the hypusine region of mature eIF-5A is directed at retroviral multiplication.

31. A method of identifying by high throughput screening a therapeutic agent that decreases the biological activity of the hypusine region of mature eIF-5A, comprising contacting hypusine-containing eIF-5A with an agent and detecting the binding of an antibody of claim 3 to hypusine-containing eIF-5A.

32. A method according to claim 31, wherein the high throughput screening of the biological activity of the hypusine region of mature eIF-5A is directed at cell proliferation.

33. A method according to claim 31, wherein the high throughput screening of the biological activity of the hypusine region of mature eIF-5A is directed a retroviral multiplication.

34. A method of identifying by high throughput screening a therapeutic agent that decreases the biological activity of the hypusine region of mature eIF-5A, comprising contacting hypusine-containing eIF-5A with an agent and detecting the binding of an antibody of claim 1 to hypusine-containing eIF-5A.

35. A method according to claim 34, wherein the high throughput screening of the biological activity of the hypusine region of mature eIF-5A is directed at cell proliferation.

36. A method according to claim 34, wherein the high throughput screening of the biological activity of the hypusine region of mature eIF-5A is directed a retroviral multiplication.

37. The method of any of claims 28, 31 or 34 comprising the steps of:
a) Preparing a quantity of purified hypusine-containing eIF-5A;
b) Attaching the purified hypusine-containing eIF-5A to a solid support;
c) Forming a reaction mixture by contacting the hypusine-containing eIF-5A of Step b with a test compound with or without antibody to hypusine-containing eIF-5A under conditions which allow binding of the test compound;
d) Washing the mixture of Step c to remove non-bound test compound;
e) Detecting the amount of hypusine-containing eIF-5A antibody bound, wherein said detecting may be accomplished by using a second antibody which is labeled with a radioactive isotope or an enzyme or chromophore; and
f) Comparing the amount of labeled second antibody bound to a sample without test compound; wherein the amount of labeled antibody bound correlates inversely with the potential of the test compound for decreasing the biological activity of the hypusine region of mature eIF-5A.

38. A method of quantifying the response to proliferation-modifying therapies, said method comprising:
a) obtaining a sample or tissue biopsy from a subject of interest prior to the administration of a proliferation modifier;
b) obtaining a sample or tissue biopsy after cessation of administration of a proliferation modifier;
c) using a ligand according to any of claims 1, 2 or 3 to measure the level of hypusine-containing antigen in said sample or tissue biopsy as reflective of the individual's response to the proliferation modifying therapy; and
wherein the proliferation modifying therapy may consist of administration of cell proliferation inhibitors, such as anti-cancer drugs, or of cell proliferation stimulators exemplified by growth hormone, erythropoietin, and similar molecules.

39. A ligand specific for the folate-binding region of eukaryotic translation initiation factor 5A, wherein said folate-binding region comprises at least one residue motif common to eIF-5A and to the bacterial and human dihydrofolate reductases as shown in FIG. 6.

40. The ligand of claim 39, wherein the ligand is selected from the group consisting of an analog of folate, derivatives thereof and fragments thereof, which specifically bind to an eIF-5A molecule only if said eIF-5A contains a folate-binding region.

41. A method for identifying folate derivatives that are inhibitors of proliferation yet do not inhibit folate-dependent enzymes, comprising placing the folate derivatives under investigation in contact with an eIF-5A molecule containing a folate-binding region, and measuring the extent, if any, to which said folate derivatives specifically bind said eIF-5A molecule.

42. The method of claim 41, wherein said folate derivatives under investigation are placed in contact with said eIF-5A molecule containing a folate-binding region, and with the ligand of claim 39, and measuring the extent to which said folate derivatives successfully compete with said ligand for binding with said eIF-5A molecule.

43. A method for inhibiting in a cell the biological activity of the folate-binding region of eIF-5A, said folate binding region comprising residue motifs as set forth in FIG. 6, comprising introducing into said cell a low-molecular weight molecule that binds to the folate-binding region of eIF-5A, and thereby inhibits the biological activity of eIF-5A required for cell proliferation.


SEQUENCE LISTING (3 sequences)

SEQ ID NO: 1 - protein, 154 aa, Homo sapiens
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 2 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGYLSLLTETGEVREDLKLPEGELGKEIEGKYNAGEDVQVSVMCAMSEEYAVAIKPCK

SEQ ID NO: 3 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND